(19) United States
(12) Patent Application Publication
(10) Pub. No.: US 2026/0308102 A1
(43) Pub. Date: Oct. 8, 2026

(54) 18F-FLUORINATED FATTY ACIDS AND USES THEREOF

(71) Applicant: Ratio Therapeutics, Inc., Boston, MA (US)

(72) Inventor: Alejandro Amor Coarasa, Woburn, MA (US)

(21) Appl. No.: 18/996,666

(22) PCT Filed: Jul. 19, 2023
(86) PCT No.: PCT/US2023/028114

§ 371 Date: Jan. 17, 2025

Related U.S. Application Data
(60) Provisional application No. 63/390,791, filed on Jul. 20, 2022.

(51) Int. Cl. C07C 69/63; A61K 51/04; A61P 35/00; C07B 59/00; C07C 53/21

(52) U.S. Cl. CPC C07C 69/63; A61K 51/0402; A61P 35/00; C07B 59/001; C07C 53/21; A61K 2121/00; C07B 2200/05

(57) ABSTRACT

Disclosed are compounds, compositions, and methods useful for imaging cells in a subject. In particular, the compound have the structure of Formula (I) is useful in the disclosed imaging methods:

### RELATED APPLICATIONS

This application claims the benefit of U.S. Provisional Application Ser. No. 63/390,791, filed Jul. 20, 2022, the entire teachings of which are incorporated herein by reference.

### BACKGROUND

Radiolabeled metabolic probes have had a fundamental role in medicine. From describing basic biological processes, to diagnosing metabolic abnormalities, to monitoring treatments, metabolic imaging agents have proven to be essential in molecular imaging. The most widely used metabolic probe is 2-deoxy-2-[¹⁸F]fluoro-D-glucose ([¹⁸F]FDG). [¹⁸F]FDG is taken up by glucose transporters (because its structure resembles the chemical structure of glucose) and metabolized incompletely (because its structure is not exactly the same as the structure of glucose), resulting in accumulation in cells imaged by positron emission tomography (PET). Ultimately, accumulation of [¹⁸F]FDG in the target tissues produces a better lesion to background ratio than ¹¹C-labeled glucose, further improving its properties as an imaging agent. Finally, a successful image with [¹⁸F]FDG is not as dependent on a precise imaging time as are other metabolizable probes. This flexibility allows clinical imaging sites to accommodate the commonplace everyday challenges which face all radiotracer imaging laboratories.

Although non-targeted cancer imaging is performed almost exclusively with [¹⁸F]FDG, glucose is not the only metabolic substrate taken up by energy-avid tumors. Rapidly growing tumors must obtain a large proportion of their lipids performed from the host. So, fatty acids (FAs), and particularly short chain FAs are important metabolic substrates that, excepting acetate, have remained largely unexplored to date.

Medium chain fatty acids (MCFAs) contain lower energy density than their long chain (LCFAs) congeners and undergo preferential β-oxidation rather than storage in adipose tissue, thereby resulting in increased post-prandial energy expenditure. In contrast, circulating LCFAs are used primarily for energy storage, but a significant percent, nonetheless, is consumed by cardiac muscle through β-oxidation. This unique characteristic of LCFAs makes them unique and important imaging agents as they target and reflect heart metabolism directly rather than blood flow as reflected by the more commonly performed perfusion scan. The major limitation suffered by LCFAs is that their oxidation occurs extremely rapidly, which makes accurate physiologic interpretation of these images quite difficult. Throughout the history of nuclear medicine, several strategies have been followed to either stop or delay β-oxidation. Among these methods include α-methylations, sulfur substitutions as well as many others. The most common radioisotopes used have included ^{123/131}I for single-photon emission computed tomography (SPECT) and planar imaging, and ¹¹C and ¹⁸F for PET imaging.

PET scanning is preferred for quantitative imaging. While ¹⁸F-labeled FAs have been largely unexplored, significant efforts have been made to produce several ¹¹C-labeled PET metabolic tracers, including FAs. Radiolabeled, unsubstituted, saturated fatty acids can be produced directly from the ¹¹CO₂ formed in the cyclotron by reaction with commercially available Grignard reagents. This is very advantageous allowing the production of large quantities of the drug product in relatively short periods of time. Furthermore, the main advantage of ¹¹C-labeled compounds lies in the biogenic nature of the radioisotope, permitting the synthesis of unmodified biological compounds, and the study of their biodistribution and metabolism. Because of the short half-life of carbon-11 (t_{1/2}=20 min), studies can be performed the same day in a single patient exposed to different conditions, or even employing different labeled probes. Interestingly, studies in cancer patients with [¹¹C]Acetate have shown that its uptake in comparison with [¹⁸F]FDG is mutually exclusive, and that the overlapping of both scans could be essential for staging and prognosis.

It must also be recognized, however, that the advantages shown by ¹¹C-labeled metabolites are accompanied by substantial limitations. From a practical perspective, the short half-life of carbon-11 requires the presence of a cyclotron and a synthesis facility on site. From the even more important perspective of pathophysiologic utility, ¹¹C-labeled probes metabolize exactly as do their stable analogues. This feature makes it impossible to know either the extent of metabolism and/or when and where that metabolic process occurs during in vivo PET imaging, which results in fundamental uncertainty as to what is being imaged. By way of contrast, probes containing longer lived isotopes and further molecular modifications have the potential to overcome these limitations. This is true not just because of their longer half-life, permitting long-distance radiotracer transport and eliminating the need for an on-site cyclotron; but also because of their in vivo behavior due to their “modified” nature.

The development of a stable, radiolabeled FA could enable early detection of well-differentiated, primary cancers that often elude detection with [¹⁸F]FDG. They could also provide diagnostic tools for cardiac metabolism, as viable PET alternative to long standing SPECT tracers like [^{99m}Tc]Sestamibi.

### SUMMARY

One aspect of the invention provides compounds, compositions, and methods useful for imaging cells in a subject.

Accordingly, provided herein is a fatty acid or fatty acid ester compound comprising a fluorine-18, e.g. a compound having the structure of Formula (I):

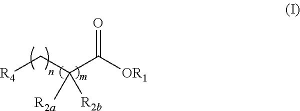

wherein
n is 0 to 15;
m is 0 or 1;
R₁ is H or alkyl;
R₂ₐ is H, alkyl, or ¹⁸F;
R_{2b} is H or alkyl;
R₄ is H or C(R₄ₐ)(R_{4b})(R_{4c}); and
R₄ₐ, R_{4b}, and R_{4c} are each independently H, alkyl, or a radioactive halogen, as describe above, such as ¹⁸F,
   provided that one and only one of R₂ₐ, R₄ₐ, R_{4b}, and R_{4c} is the radioactive halogen such as ¹⁸F.
or a salt thereof,
   provided the compound is not [¹⁸F]-fluoroacetate or 2-[¹⁸F]fluoropropionate, or a salt thereof.

Another aspect of the invention provides compounds, compositions, and methods useful for imaging cellular uptake of a fatty acid in a subject.

Another aspect of the invention provides compounds, compositions, and methods useful for imaging β-oxidation of a fatty acid in a subject.

Another aspect of the invention is a fatty acid or fatty acid ester compound comprising a halogen, e.g., a radioactive halogen such as ¹³¹I, ¹²³I, ¹²⁵I, ¹²⁴I, ⁷⁶Br, ⁷⁷Br or ¹⁸F. In one embodiment, the radioactive halogen is fluorine-18.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the disclosure will be apparent from the detailed description, and from the claims.

### BRIEF DESCRIPTION OF THE DRAWINGS

FIG. 1 is a series of Static Sagital and Coronal Maximum Intensity Projections (MIPs) μPET/CT images obtained at 45 minutes p.i. for the compounds studied (excluding palmitate) in normally fed animals. Imaging with ethyl-protected intermediates are also shown (e-). FIG. 1 also contains a graph depicting the results from organ biodistribution studies performed at 45 minutes p.i. for 2-[¹⁸F]FB and 2-[¹⁸F]FP in similar conditions.

FIG. 2 is a graph depicting PET-derived quantification of brain and pancreatic (“gut”) uptake of 2-[¹⁸F]FP and 2-(L)-[¹⁸F]FP under different conditions (n=2). Co-injection with AZD-3965 caused significant blocking of fatty acid uptake and increased excretion.

FIG. 3 is a series of Static Sagital and Coronal Maximum Intensity Projections (MIPs) μPET/CT images obtained at 45 minutes p.i. for MCFAs of chain lengths C-5 to C-8. Influence of fasting (or lack thereof) vs. feeding is shown in the images for the 2-substituted MCFAs.

FIG. 4 is a series of Static Sagital and Coronal Maximum Intensity Projections (MIPs) μPET/CT images obtained at 45 minutes p.i. for 16- and 2-[¹⁸F]FPalm, including fasting for 2-[¹⁸F]FPalm (a 16 carbon chain FA).

FIG. 5 is a graph showing image-derived organ contents for all studied compounds (n=2 per compound) at 45 min p.i., including the previously published short chain FAs.

FIG. 6 is a series of graphs and associated images showing the pharmacokinetics of 2-[¹⁸F]FP, 2-[¹⁸F]FIB and [¹⁸F]FDG in a SKOV3 tumor model.

FIG. 7 is a graph showing the organ biodistribution of 2-[¹⁸F]FIB (n=3).

FIG. 8 is a collection of dynamic μPET/CT images depicting cardiac uptake and metabolism of n-substituted FAs in Sprague Dawley Rats. Almost complete defluorination was observed at 45 minutes (free ¹⁸F bone uptake), while reduced and sometimes absent metabolism was observed for the 2-substituted compounds.

FIG. 9 is a graph of p-MOD analysis showing evidence of high brain uptake of the studied 2-substituted FAs at 45-60 min post administration. All animals were continuously fed with the exception of [¹⁸F]FDG, which needed fasting.

FIG. 10 is a series of dynamic μPET/CT images depicting the biodistribution of 2-[¹⁸F]FB and 4-[¹⁸F]FB in wild type mice. Stable biodistribution was achieved after 45 minutes p.i.

FIG. 11 is a collection of dynamic μPET/CT images comparing the biodistribution of 2-[¹⁸F]FB and 4-[¹⁸F]FB between wild type mice that were fed or fasted overnight.

FIG. 12 is a collection of dynamic μPET/CT images that compare different imaging conditions including blocking with the MCT1 blocker AZD3965.

### DETAILED DESCRIPTION

Described herein are 18-F fluorinated fatty acids and fatty acid esters. In certain embodiments, the compounds mimic the in vivo behavior of their biogenic congeners, while failing to undertake regular metabolic pathways and therefore accumulate in target tissues as [¹⁸F]FDG does.

### Definitions

For convenience, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

Certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds may exist in particular geometric or stereoisomeric forms. The present disclosure contemplates all such compounds, including cis- and trans-isomers, R- and S-enantiomers, diastereomers, (D)-isomers, (L)-isomers, the racemic mixtures thereof, and other mixtures thereof, as falling within the scope of the disclosure. Additional asymmetric carbon atoms may be present in a substituent such as an alkyl group. All such isomers, as well as mixtures thereof, are intended to be included in this disclosure.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The disclosure embraces all of these forms.

Structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this disclosure.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions are non-pyrogenic, i.e., do not induce significant temperature elevations when administered to a patient.

In some embodiments, salt refers to a pharmaceutically acceptable salt. The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods described herein may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid coformers that do not form formal ionic interactions with the small molecule.

An “effective amount” of a compound with respect to use in any of the imaging methods discloses here, refers to an amount of the compound in a preparation which, when administered as part of a desired imaging regimen (to a mammal, preferably a human) results in accumulation of an amount of the compound in the subject by a desired imaging technique, e.g. PET imaging or PET/CT imaging.

The term “patient” or “subject” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer. Alkyl groups may be substituted or unsubstituted. In some embodiments, an alkyl group is linear or branch and has from 1-30, 1-20, 1-15, 1-10, 1-5, 10-30, 15-30 or 20-30 carbon atoms.

As used herein, the term “haloalkyl” refers to an alkyl group as hereinbefore defined substituted with at least one halogen.

As used herein, the term “fluoroalkyl” refers to an alkyl group as hereinbefore defined substituted with at least one fluoro.

As used herein, the term “alkylene” refers to an alkyl group having the specified number of carbons, for example from 2 to 12 carbon atoms, that contains two points of attachment to the rest of the compound on its longest carbon chain. Non-limiting examples of alkylene groups include methylene —(CH₂)—, ethylene —(CH₂CH₂)—, n-propylene-(CH₂CH₂CH₂)—, isopropylene —(CH₂CH(CH₃))—, and the like. Alkylene groups can be cyclic or acyclic, branched or unbranched carbon chain moiety, and may be optionally substituted with one or more substituents.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the definition of each expression, e.g., alkyl, m, n, etc., when it occurs more than once in any structure, is intended to be independent of its definition elsewhere in the same structure.

As used herein, “small molecules” refers to small organic or inorganic molecules of molecular weight below about 3,000 Daltons. In general, small molecules have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs encompass oligopeptides and other biomolecules having a molecular weight of less than about 1000.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

A “radiopharmaceutical agent,” as defined herein, refers to a pharmaceutical agent which contains at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

“In one aspect, the disclosed compounds have a radioactivity level of between 20 mCi and 100 mCi. In another aspect, the disclosed compounds have a radioactivity level of between 100 mCi and 500 mCi. In another aspect, the disclosed compounds have a radioactivity level of between 250 mCi and 2500 mCi. In another aspect, the disclosed compounds have a radioactivity level of between 500 mCi and 5000 mCi. In another aspect, the disclosed compounds have a radioactivity level of between 20 mCi and 500 mCi. In another aspect, the disclosed compounds have a radioactivity level of between 20 mCi and 2500 mCi. In another aspect, the disclosed compounds have a radioactivity level of between 20 mCi and 5000 mCi. In another aspect, the disclosed compounds have a radioactivity level of between 100 mCi and 2500 mCi. In another aspect, the disclosed compounds have a radioactivity level of between 100 mCi and 5000 mCi. In another aspect, the disclosed compounds have a radioactivity level of between 250 mCi and 5000 mCi.”

“Short-chain fatty acids (SCFA),” as defined herein, refer to are fatty acids with aliphatic tails of five or fewer carbons (e.g. butyric acid).

“Medium-chain fatty acids (MCFA),” as defined herein, refer to fatty acids with aliphatic tails of 6 to 12 carbons, which can form medium-chain triglycerides.

“Long-chain fatty acids (LCFA),” as defined herein, refer to fatty acids with aliphatic tails of 13 to 21 carbons.

“Very long chain fatty acids (VLCFA),” as defined herein, refer to fatty acids with aliphatic tails of 22 or more carbons.

For purposes of this disclosure, the chemical elements are identified in accordance with the Periodic Table of the Elements, CAS version, Handbook of Chemistry and Physics, 67th Ed., 1986-87, inside cover.

### Compounds

One aspect of the invention relates to a fatty acid or fatty acid ester compound comprising a fluorine-18, provided the compound is not [18F]-fluoroacetate or 2-[18F]fluoropropionate, or a salt thereof.

In certain embodiments, the fatty acid or salt thereof or fatty acid ester is a saturated fatty acid or salt thereof or fatty acid ester substituted with a fluorine-18.

In certain embodiments, the fatty acid or salt thereof or fatty acid ester is a branched fatty acid or salt thereof or fatty acid ester substituted with a fluorine-18.

In certain embodiments, the fatty acid or salt thereof or fatty acid ester is a short-chain fatty acid or salt thereof or fatty acid ester. In other embodiments, the fatty acid or salt thereof or fatty acid ester is a medium-chain fatty acid or salt thereof or fatty acid ester. In other embodiments, the fatty acid or salt thereof or fatty acid ester is a long-chain fatty acid or salt thereof or fatty acid ester. In other embodiments, the fatty acid or salt thereof or fatty acid ester is a very long-chain fatty acid or salt thereof or fatty acid ester.

In certain embodiments, the carbon alpha to the carbonyl is substituted with a fluorine 18. In other embodiments, the terminal methyl is substituted with a fluorine 18.

One aspect of the invention relates to a compound having the structure of Formula (I):

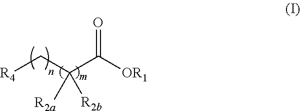

wherein
n is 0 to 15;
m is 0 or 1;
R₁ is H or alkyl;
R₂ₐ is H, alkyl, or ¹⁸F;
R_{2b} is H or alkyl;
R₄ is H or C(R₄ₐ)(R_{4b})(R_{4c}); and
R₄ₐ, R_{4b}, and R_{4c} are each independently H, alkyl, or ¹⁸F,
   provided that one and only one of R₂ₐ, R₄ₐ, R_{4b}, and R_{4c} is ¹⁸F, provided the compound is not [18F]-fluoroacetate or 2-[18F]fluoropropionate, or a salt thereof.

In some embodiments, the compound is selected from the following Table 1:

TABLE 1

| | |
|---|---|
| e-[¹⁸F]FA | 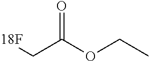 |
| e-2-[¹⁸F]FP | 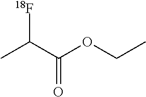 |
| e-3-[¹⁸F]FP | 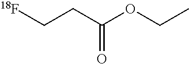 |
| 3-[¹⁸F]FP | 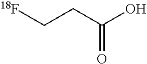 |
| e-2-[¹⁸F]FIB | 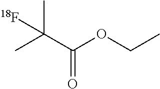 |
| 2-[¹⁸F]FIB | 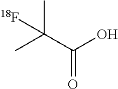 |
| e-3-[¹⁸F]FB | 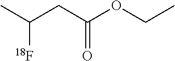 |
| 3-[¹⁸F]FB | 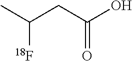 |
| e-4-[¹⁸F]FB | 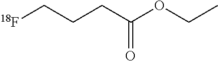 |
| 4-[¹⁸F]FB | 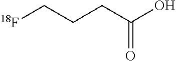 |
| e-3-[¹⁸F]FIB | 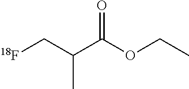 |
| 3-[¹⁸F]FIB | 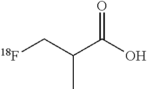 |
| e-2-[¹⁸F]FB | 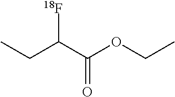 |
| 2-[¹⁸F]FB | 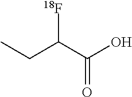 |
| e-2-(L)-[¹⁸F]FP | 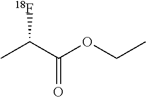 |
| 2-(L)-[¹⁸F]FP | 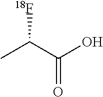 |
| e-2-[¹⁸F]FV | 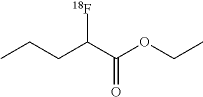 |
| 2-[¹⁸F]FV | 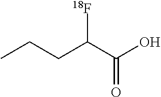 |
| e-5-[¹⁸F]FV | 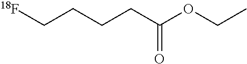 |
| 5-[¹⁸F]FV | 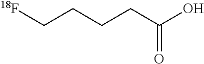 |
| e-2-[¹⁸F]FC | 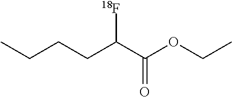 |
| 2-[¹⁸F]FC | 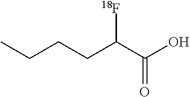 |
| e-6-[¹⁸F]FC | 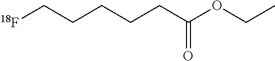 |
| 6-[¹⁸F]FC | 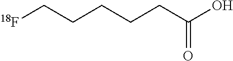 |
| e-2-[¹⁸F]FE | 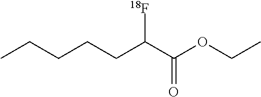 |
| 2-[¹⁸F]FE | 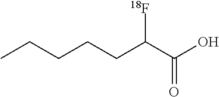 |
| e-7-[¹⁸F]FE | 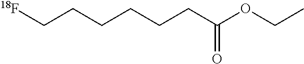 |
| 7-[¹⁸F]FE | 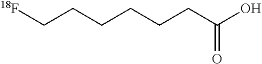 |
| e-2-[¹⁸F]FCy | 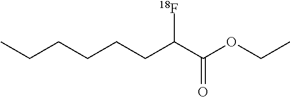 |
| 2-[¹⁸F]FCy | 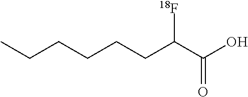 |
| e-8-[¹⁸F]FCy | 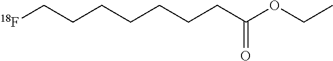 |
| 8-[¹⁸F]FCy | 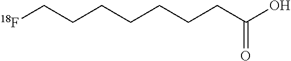 [SMILES: O=C(O)CCCCCCC[18F]] |
| ethyl 2- [¹⁸F]Fluorohexadecanoate | 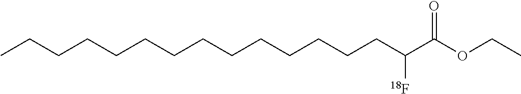 [SMILES: CCCCCCCCCCCCCCC([18F])C(=O)OCC] |
| 2- [¹⁸F]Fluorohexadecanoic acid | 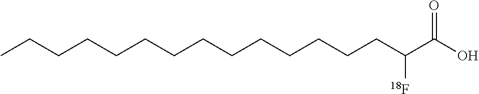 [SMILES: CCCCCCCCCCCCCCC([18F])C(=O)O] |
| methyl 16- [¹⁸F]Fluorohexadecanoate | 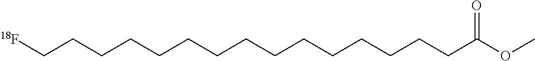 [SMILES: COC(=O)CCCCCCCCCCCCCCC[18F]] |
| ethyl 16- [¹⁸F]Fluorohexadecanoate | 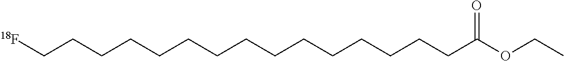 [SMILES: CCOC(=O)CCCCCCCCCCCCCCC[18F]] |
| 16- [¹⁸F]Fluorohexadecanoic acid | 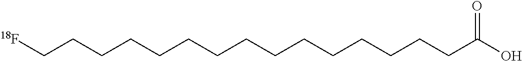 [SMILES: O=C(O)CCCCCCCCCCCCCCC[18F]] |

Salts of the compounds in Table 1 are also included.

In certain embodiments, the compound relates to any of the compounds described herein, provided the compound is not 3-[18F]fluoropropionate, ethyl 3-[18F]fluoropropionate, 3-[18F]fluorobutyrate, ethyl 3-[18F]fluorobutyrate, 3-[18F]fluoroisobutyrate, or ethyl 3-[18F]fluoroisobutyrate.

In some embodiments, the compounds are atropisomers. Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this disclosure. Such compounds are useful, for example, as analytical tools, as probes in biological assays, or as therapeutic agents in accordance with the present disclosure. For example, in the case of variable R¹, the (C₁-C₄)alkyl or the —O—(C₁-C₄)alkyl can be suitably deuterated (e.g., —CD₃, —OCD₃). Also, compounds produced by the replacement of a fluorine with an ¹⁸F enriched fluorine are within the scope of this disclosure.

Any compound can also be radiolabel for the preparation of a radiopharmaceutical agent.

### Methods of Treatment

One aspect of the invention relates to a method of imaging target cells in a subject comprising:
(i) introducing into the subject an effective amount of a compound described herein or salt thereof, wherein the compound specifically or preferentially accumulates in the target cells of the subject;
(ii) detecting in the subject the location of the compound in the subject; and
(iii) obtaining one or more images of the target cells in the subject based on the location of the compound in the subject.

In certain embodiments, the ¹⁸F in the compound is detected by positron emission tomography (PET).

In certain embodiments, the target cell is a cancer cell, a cardiac muscle cell, a nerve cell, or a glial cell.

In certain embodiments, the method further comprises (iv) determining whether the subject is afflicted with a cardiovascular disease, a neurodegenerative disease, or a cancer based on an analysis of the one or more images of the target cells.

Another aspect of the invention relates to a method of imaging cellular uptake of a fatty acid in a target cell of a subject comprising:
(i) introducing into the subject an effective of a compound described herein or salt thereof;
(ii) performing one or more positron emission tomography (PET) scans of the subject to detect the compound in the target cell; and
(iii) obtaining one or more images of the target cell based on the location of the compound in the subject.

Another aspect of the invention relates to a method of imaging β-oxidation of a fatty acid in a target cell of a subject comprising:
(i) introducing into the subject an effective amount of a compound described herein or salt thereof;
(ii) performing one or more positron emission tomography (PET) scans of the subject to detect the compound in the target cell; and
(iii) obtaining one or more images of the target cell based on the location of the compound in the subject.

In certain embodiments, the ¹⁸F in the compound is detected by the positron emission tomography (PET).

In certain embodiments, the target cell is a cancer cell, a cardiac muscle cells, a nerve cell, or a glial cell.

In certain embodiments, the one or more positron emission tomography (PET) scans of the subject are performed on a tissue of interest in the subject.

In certain embodiments, the tissue of interest is cardiac tissue. In other embodiments, the tissue of interest is brain tissue. In other embodiments, the tissue of interest is brain, breast, lung, kidney, gastrointestinal, pancreatic, prostate, or spinal cord tissue.

In certain embodiments, the cardiac tissue is afflicted with a cardiovascular disease.

In certain embodiments, the brain tissue is afflicted with a neurodegenerative disease.

In certain embodiments, the brain, breast, lung, kidney, gastrointestinal, pancreatic, prostate, or spinal cord tissue is afflicted with a cancer or tumor.

In certain embodiments, the tissue of interest is healthy tissue.

In certain embodiments of any one of the disclosed methods, the compound of Formula (I) is defined as:

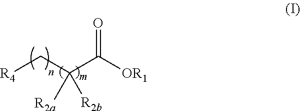

wherein
n is 0 to 15;
m is 0 or 1;
R₁ is H or alkyl;
R₂ₐ is H, alkyl, or ¹⁸F;
R_{2b} is H or alkyl;
R₄ is H or C(R₄ₐ)(R_{4b})(R_{4c}); and
R₄ₐ, R_{4b}, and R_{4c} are each independently H, alkyl, or ¹⁸F,
   provided that one and only one of R₂ₐ, R₄ₐ, R_{4b}, and R_{4c} is a ¹⁸F,
or a salt thereof,
   provided the compound is not [18F]-fluoroacetate or 2-[18F]fluoropropionate, or a salt thereof.

In certain embodiments of any one of the disclosed methods, wherein in the compound of Formula (I), n is 2-4 and m is 1, or n is 1-4 and m is 0.

### Pharmaceutical Compositions, Routes of Administration, and Dosing

In certain embodiments, the invention is directed to a composition, comprising a compound described herein and an acceptable carrier. In certain embodiments, the composition comprises a plurality of compounds described herein and an acceptable carrier.

In certain embodiments, the invention is directed to a pharmaceutical composition, comprising a compound described herein and a pharmaceutically acceptable carrier. In certain embodiments, the pharmaceutical composition comprises a plurality of compounds described herein and a pharmaceutically acceptable carrier.

In certain embodiments, intravenous administration of a compound may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

Generally, daily oral doses of a compound will be, for human subjects, from about 0.01 milligrams/kg per day to 1000 milligrams/kg per day. It is expected that oral doses in the range of 0.5 to 50 milligrams/kg, in one or more administrations per day, will yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the effective amount can be initially determined from animal models. An effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations described herein can be administered in pharmaceutically acceptable solutions, which may routinely contain pharmaceutically acceptable concentrations of salt, buffering agents, preservatives, compatible carriers, adjuvants, and optionally other therapeutic ingredients.

For use in imaging, an effective amount of the compound can be administered to a subject by any mode that delivers the compound to the desired target cells or tissue of interest. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the disclosure can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol moieties are suitable.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The compound can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro) capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compounds described herein and optionally other therapeutics may be administered per se (neat) or in the form of a pharmaceutically acceptable salt or cocrystal. When used in medicine the salts or cocrystals should be pharmaceutically acceptable, but non-pharmaceutically acceptable salts or cocrystals may conveniently be used to prepare pharmaceutically acceptable salts or cocrystals thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions described herein contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds described herein, and with each other, in a manner such that there is no interaction which would substantially impair the desired pharmaceutical efficiency.

The agent(s), including specifically but not limited to a compound described herein, may be provided in particles. Particles as used herein means nanoparticles or microparticles (or in some instances larger particles) which can consist in whole or in part of the compound or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodable, biodegradable, or nonbiodegradable material or combinations thereof. The particles may be microcapsules which contain the compound described herein in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

It will be understood by one of ordinary skill in the relevant arts that other suitable modifications and adaptations to the compositions and methods described herein are readily apparent from the description contained herein in view of information known to the ordinarily skilled artisan, and may be made without departing from the scope of the disclosure or any embodiment thereof. Having now described the present invention in detail, the same will be more clearly understood by reference to the following examples, which are included herewith for purposes of illustration only and are not intended to be limiting of the invention.

The invention is further described in the following examples, which do not limit the scope of the invention described in the claims.

### EXEMPLIFICATION

### Example 1: [¹⁸F]Labeled Short Chain Fatty Acids for PET Imaging

### Materials and Methods

### Precursors and Standards:

All precursors were purchased from commercial vendors. Standards and radiolabeled compounds were synthesized using the same procedures, namely via nucleophilic substitution and subsequent acid hydrolysis. For preparation of the standards, 5 mg of [¹⁹F]KF were spiked with 740-1850 MBq (20-50 mCi) of fluorine-18, and the radio-peak was used for identification and collection of the product as done in a regular radiosynthesis. After complete fluorine-18 decay, the next day the samples were frozen, lyophilized and redissolved in proper solvents (CD₃—CN for esters and D₂O for acids) for NMR analysis.

### Radiolabeling Strategy:

Radiolabeling was performed by nucleophilic substitution of Bromine containing fatty acids. Briefly, [¹⁸F]F⁻ in [¹⁸O]H₂O (no carrier added) was received from the cyclotron (NCM USA, Bronx, NY), to which 1 ml of an 80% MeCN:H₂O solution (v:v) containing 10 mg of Kryptofix (K₂₂₂) and 5 mg of K₂CO₃ were added. The mixture was heated at 95 degrees Celsius under a light N₂ stream and house vacuum with a cold trap while shaking. When dried, two subsequent additions of 1 ml of extra dry AcroSeal® (Acros Organics, USA) were made with an insulin syringe (BD Micro-Fine™, USA) under the same N₂ flow, vacuum and continuous heating. Once dried, the N₂ and vacuum needles were withdrawn from the silicon septum on the sealed Reacti-Vial (Pierce, USA). To this vial, 0.6 ml of a solution containing 10 mg/ml of the desired brominated precursor in dry MeCN were added and the reaction was held for 10 minutes (Scheme 1). When preparing the esters, this solution was injected into a Semi-Prep HPLC system (Varian-Agilent, USA) with a Water/MeCN 0.05% TFA binary system. The products were collected and reformulated by trapping in an HLB Plus or C-18 Plus Sep-Pak (Waters, USA), and eluting with a combination of EtOH and saline to form a <10% EtOH saline containing injectable solution, as necessary.

When preparing the FAs, hydrolysis was studied with both NaOH and HCl at different concentrations (Scheme 1). The resulting hydrolyzed solutions containing FAs were injected into a Semi-Prep HPLC system with a PRP-1 (10×250 mm) polymeric reverse phase column and a Saline/EtOH binary system that allowed collection of injectable solutions directly for further experimentation. Radiolabeled esters' quality control was performed in an analytical HPLC with a reverse phase C-18 column (4.6×50 mm) and a H₂O/MeCN (0.05% TFA) 10-minute gradient. Radiolabeled FAs were analyzed at 1 ml/min while their corresponding esters at 2 ml/min flowrate. Since these syntheses were used only for animal experiments, the 0.22 μm filters were assumed integral and not bubble point tested. Endotoxins were tested by limulus amoebocyte lysate (LAL) clothing with a limit of 0.05 EU/ml.

Finally, to prepare enantiomerically pure 2-(L)-[¹⁸F]FP, commercially available (Sigma, USA) ethyl(2R)-2-{[(4-methylphenyl) sulfonyl]oxy}propanoate was used as precursor. The procedure was identical to the one described above but the temperature to produce the e-2-(L)-[¹⁸F]FP intermediary was reduced to 80° C. to avoid elimination. A detailed schematic synthesis can be found in Scheme 1 and the obtained synthetic yield in Table 2.

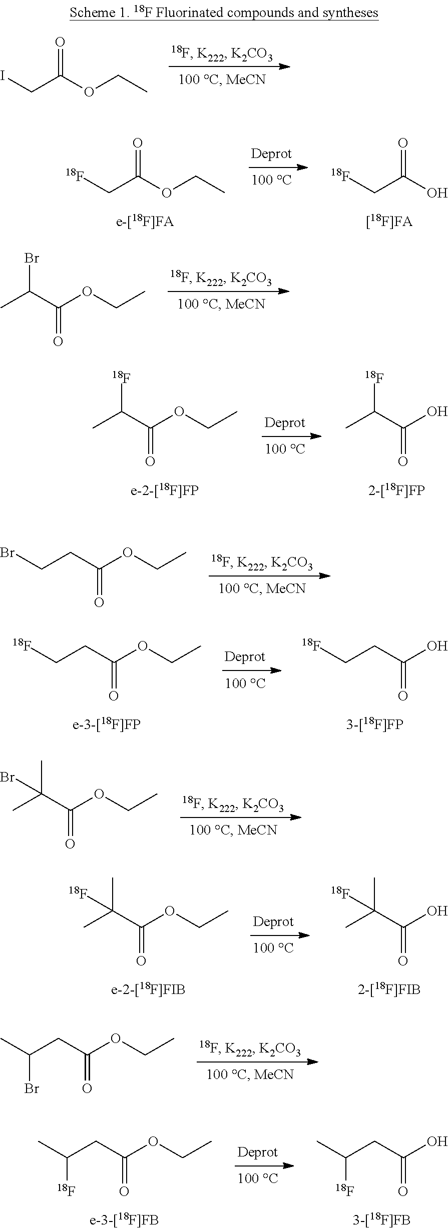

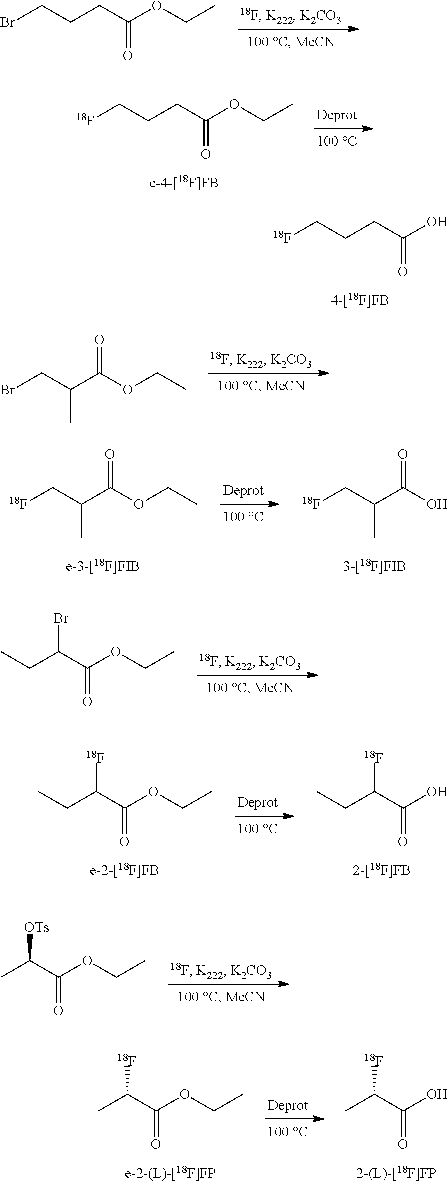

### Animal Experiments:

All animal experiments were approved by the Albert Einstein College of Medicine IACUC. Wild type immunocompetent animals were used, both for imaging and biodistribution experiments.

### Dynamic PET Imaging:

Animals were anesthetized under 3% isoflurane in 02. The solution containing the high specific activity radiolabeled compounds (9.25-18.5 MBq, or 250-500 μCi, n=2-3) was administered via retroorbital injection and the animals (n=2-3) were immediately placed in the camera where a 60 minute dynamic PET scan was performed first, followed by a 5-minute CT. Animals were allowed to recover from anesthesia and were carefully observed before moving them back to the holding facility for decay. Data was reconstructed as five 12-minute frames and as twelve 5-minute frames for analysis.

### Static PET Imaging:

The activity was administered to the awake animals (n=2) via tail-vein injection (9.25-18.5 MBq, or 250-500 μCi) and placed back into their cages. At 35 minutes post injection (p.i.), animals were place in an acrylic enclosure under 4% isoflurane anesthesia in O₂ for initial knockdown, then reduced to 2.5%. Once asleep, animals were placed in the imaging holder and a 5-minute CT was followed by a 15-minute static PET (approx. 45-60 min p.i., or 10-15 min after completion of anesthesia). As before, animals were allowed to recover from the anesthesia before returning to the holding facility for decay. When warranted, animals were studied with regular feeding and fasting for 6-12 hours to evaluate any difference in biodistribution.

### Organ Biodistribution Studies:

Biodistribution studies were performed only for 2-[¹⁸F]FB and 2-[¹⁸F]FP. As before, the activity was administered via tail-vein (1.5 MBq, 40 μCi, n=3-4) and animals were placed back in their respective cages. At 40 minutes p.i., animals were placed into an acrylic enclosure and anesthetized with 4% isoflurane in O₂ for 5 minutes. At 45 minutes p.i. animals were euthanized by neck dislocation under isoflurane. Organs including blood, heart, lungs, liver, intestines (with contents) spleen, kidneys, bone, muscle, and brain were collected, weighed and counted in an automated well counter. Results are expressed as percent of injected activity per gram of tissue (% IA/g).

### Results

All syntheses produced a 71.5±11.2% decay corrected yield (n=52 total, 1≤n≥7 per compound) in a 45 minute non-optimized, one-pot synthetic scheme. Detailed data for each synthesis is presented in Table 2. All products were successfully identified with their stable analogues by HPLC UV co-registration at 220 nm.

All attempts to perform a basic hydrolysis with NaOH in any of these compounds resulted in immediate substantial fluoride elimination, which was not observed (or was minor) when performing acid hydrolysis. When studying acid hydrolyses, it was also found that concentrations of acid greater than 6 M also cause significant fluoride elimination, and a smaller than 4 M solution will need more than 20 minutes to complete. When testing a 6 M HCl solution, deprotection was partially achieved (≈80%) at 5 minutes in 95 degrees Celsius and was completed (>95%) in 10 minutes. Therefore, all compounds were hydrolyzed by adding 0.6 ml of a 6M HCl solution to the reaction mixture, and no attempt was made to perform individual optimizations.

Although structures e-3-[¹⁸F]FB, 3-[¹⁸F]FB, e-3-[¹⁸F]FP, and 3-[¹⁸F]FP were prepared with radioactive ¹⁸F and a corresponding radio-peak was observed for both protected and hydrolyzed forms, these were not stable at HCl concentrations lower than 3 M and decomposed immediately in saline solution. Preparations e-3-[¹⁸F]FIB and 3-[¹⁸F]FIB were halted by unstable intermediates, producing only 2.2% (by HPLC) immediately after the fluorination step and defluorination after 10 minutes. All other compounds (including those with racemic mixtures) were prepared successfully, and a compendium of the synthetic results is detailed in Table 2.

TABLE 2
Synthetic results.

| | | | | | |
|---|---|---|---|---|---|
| [¹⁸F]FA | [¹⁸F]Fluoroacetate | C₂H₃FO₂ | 48.1 ± 10.0 | 99.6 ± 0.1 | 2 |
| e-[¹⁸F]FA | Ethyl [¹⁸F]Fluoroacetate | C₄H₇FO₂ | 78.5 ± 7.2 | 99.7 ± 0.5 | 2 |
| 2-[¹⁸F]FP | 2-[¹⁸F]Fluoropropionate | C₃H₅FO₂ | 74.6 ± 9.0 | 99.9 ± 0.2 | 9 |
| e-2-[¹⁸F]FP | Ethyl 2- | C₅H₉FO₂ | 79.5 ± 7.7 | 99.9 ± 0.1 | 4 |
| | [¹⁸F]Fluoropropionate | | | | |
| 3-[¹⁸F]FP | 3-[¹⁸F]Fluoropropionate | C₃H₅FO₂ | 10.6*^{#} | 0.0^{#} | 1 |
| e-3-[¹⁸F]FP | Ethyl 3- | C₅H₉FO₂ | 10.1*^{#} | 0.0^{#} | 1 |
| | [¹⁸F]Fluoropropionate | | | | |
| 2-[¹⁸F]FB | 2-[¹⁸F]Fluorobutyrate | C₄H₇FO₂ | 70.1 ± 9.9 | 99.9 ± 0.1 | 9 |
| e-2-[¹⁸F]FB | Ethyl 2- | C₆H₁₁FO₂ | 85.2 ± 18.3 | 99.9 ± 0.1 | 5 |
| | [¹⁸F]Fluorobutyrate | | | | |
| 3-[¹⁸F]FB | 3-[¹⁸F]Fluorobutyrate | C₄H₇FO₂ | 28.8 ± 20.3^{#} | 0.0^{#} | 3 |
| e-3-[¹⁸F]FB | Ethyl 3- | C₆H₁₁FO₂ | 47.0 ± 32.6^{#} | 0.0^{#} | 3 |
| | [¹⁸F]Fluorobutyrate | | | | |
| 4-[¹⁸F]FB | 4-[¹⁸F]Fluorobutyrate | C₄H₇FO₂ | 63.2 ± 15.3 | 99.7 ± 0.6 | 6 |
| e-4-[¹⁸F]FB | Ethyl 4- | C₆H₁₁FO₂ | 75.5 ± 15.9 | 99.9 ± 0.1 | 4 |
| | [¹⁸F]Fluorobutyrate | | | | |
| 2-[¹⁸F]FIB | 2-[¹⁸F]Fluoroisobutyrate | C₄H₇FO₂ | 65.5 ± 14.8 | 99.9 ± 0.1 | 6 |
| e-2-[¹⁸F]FIB | Ethyl 2- | C₆H₁₁FO₂ | 81.3 ± 3.3 | 99.9 ± 0.1 | 2 |
| | [¹⁸F]Fluoroisobutyrate | | | | |
| 3-[¹⁸F]FIB | 3-[¹⁸F]Fluoroisobutyrate | C₄H₇FO₂ | 0.0*^{#} | 0.0^{#} | 1 |
| e-3-[¹⁸F]FIB | Ethyl 3- | C₆H₁₁FO₂ | 2.3*^{#} | 0.0^{#} | 1 |
| | [¹⁸F]Fluoroisobutyrate | | | | |
| 2-(L)-[¹⁸F]FP | 2-(L)-[¹⁸F]Fluoropropionate | C₃H₅FO₂ | 42.5^{#} | 99.6^{#} | 1 |
| TOTALS | | | 72.1 ± 11.1 | 99.8 ± 0.2 | 60 |

DCRCY: Decay corrected radiochemical yield, and RCP: Radiochemical purity.
*not isolated, detected by Radio-HPLC.
^{#}Not included in the averaged totals.

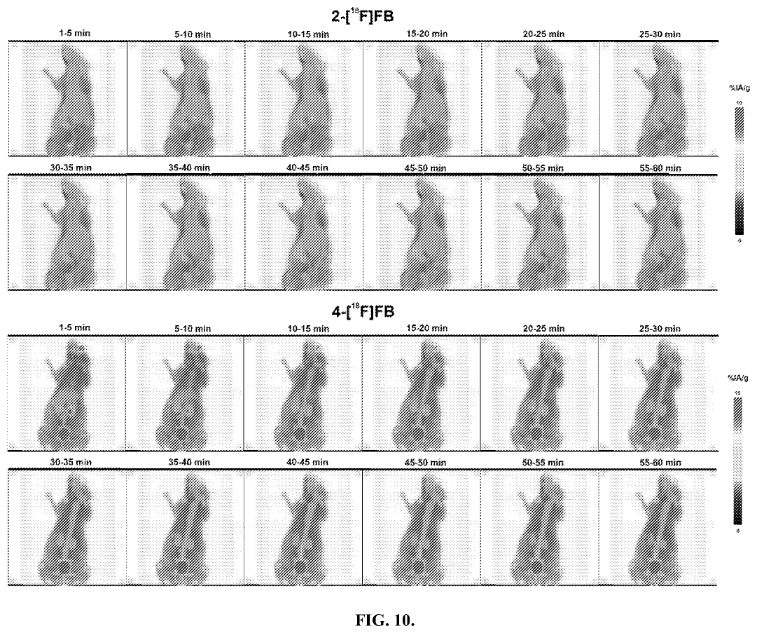

All purified products (n=42) had an average radiochemical purity of 99.8±0.2 (Table 2), a chemical purity greater than 95% (by HPLC UV at 220 nm) and a molar activity greater than 5 Ci/μmol. Compound purity remained unchanged while kept in the injectable solution at room temperature for over 4 hours after the end of synthesis for all isolated products. The sterile, pyrogen-free and isotonic products collected from the Saline/EtOH HPLC system or reformulation were filtered through a 0.2 μm membrane for injection into the animals. Initial dynamic PET studies established that full biodistribution was achieved at 45 minutes post injection FIG. 10).

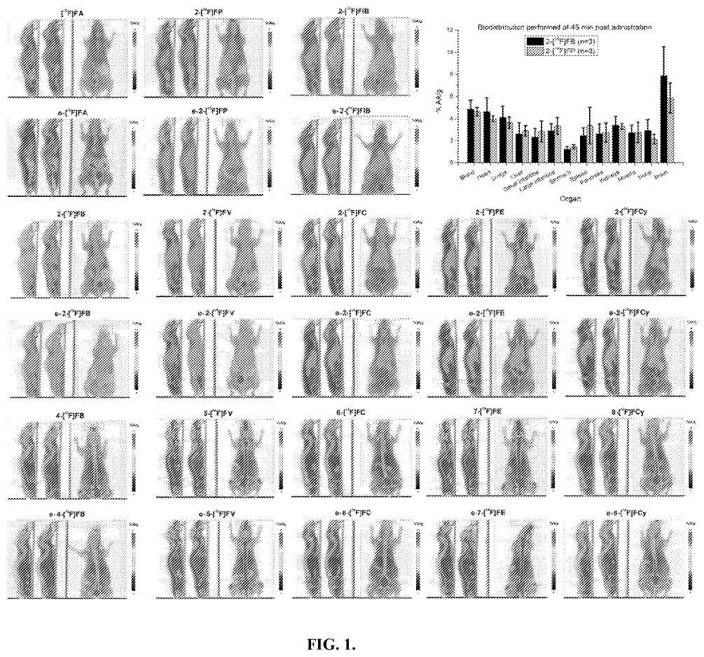

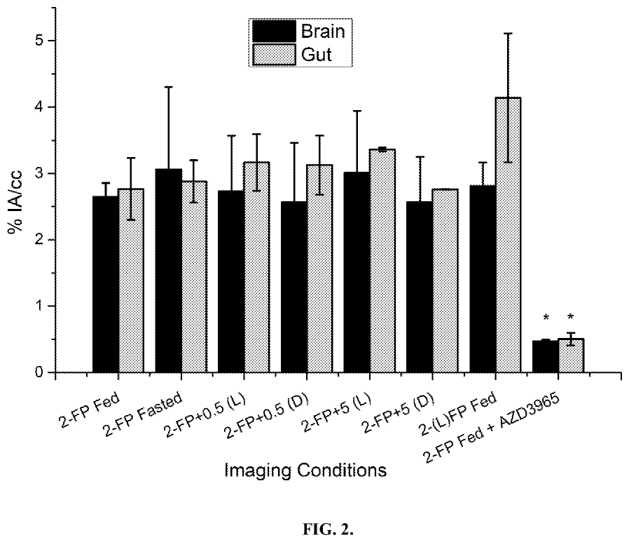

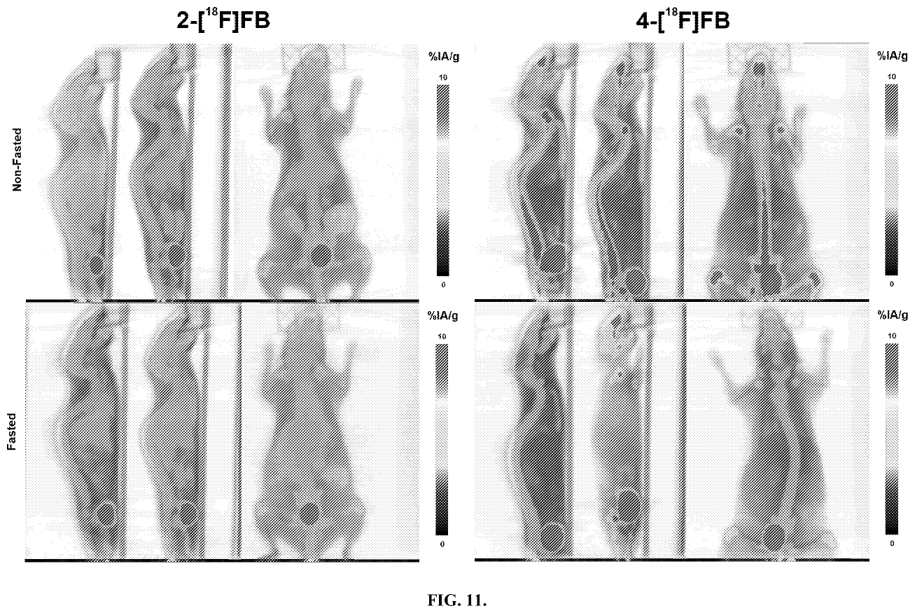

Static imaging at 45-minute pi. with fasted and fed animals, also performed with both 2-[¹⁸F]FB and 4-[¹⁸F]FB, showed some metabolic differences with reduced defluorination. This reduction in metabolic activity was especially noticeable for 4-[¹⁸F]FB. Interestingly 2-[¹⁸F]FB had a modest reduction in what seem to be an undefined and specific gut uptake that could not be identified during organ biodistributions, suggesting a transience possibly due to free motility and/or reabsorption. A small reduction in brain uptake (not significant) was also observed, perhaps due to the increase of native fatty acids and ketone bodies when fasting or just due to normal experimental variability (FIG. 11). Although intensity differences were observed, the overall biodistribution of the tracers did not change dramatically. Therefore, we proceeded with our imaging screening in fed conditions, which simplified the overall process taking into consideration the number of compounds tested (FIG. 1). Organ biodistributions of 2-[¹⁸F]FB and 2-[¹⁸F]FP were conducted to confirm the imaging results. Unfortunately, the source of the specific gut uptake could not be identified when measuring the intestines with their contents. We suspect that this gut uptake is related to the intestinal bacterial flora, known to consume fatty acids avidly, not confirmed with our experiments, but selectively blocked with AZD-3965 (FIG. 2).

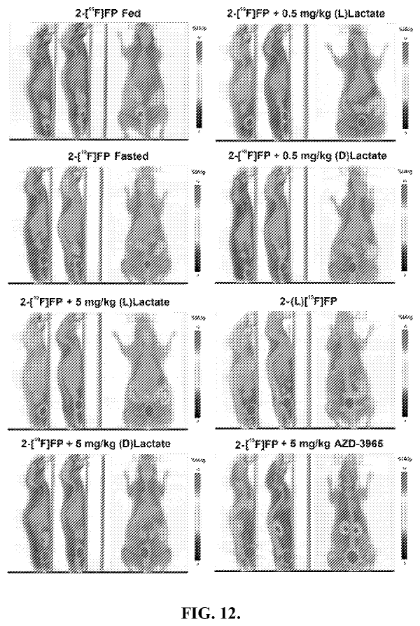

Further experiments to test the possibility of using 2-[¹⁸F]FP and 2-(L)-[¹⁸F]FP as a lactate surrogate, including co-injection with (L)Lactate, (D)Lactate, and AZD-3965 to produce displacement and monocarboxylate transporter (MCT) inhibition (respectively) were conducted and results can be observed in FIG. 2. The corresponding μPET/CT images can be found in the FIG. 12.

### Discussion

### Radiolabeling Strategy:

The synthetic strategy was designed to enable easy automation. The strategy was to produce these compounds in high yields, while maintaining simplicity as well as automation capabilities and low cost. Since keeping the amount of alkali as low as possible is important to avoid fluoride elimination, a 5/10 mg/ml K₂CO₃/K₂₂₂ system was used, which was previously identified as the lowest amount of base that can completely elute a fluorine loaded QMA cartridge. It was decided to use nucleophilic substitution of brominated precursors because their HPLC purification using PRP-1 columns was relatively easy. The elimination of the brominated acids and esters via HPLC purification is perhaps unnecessary since the high specific activities here obtain are seldom needed for metabolic probes. Toxicity is also unlikely given the high yields here reported, which could enable producing a drug product containing 10 μg or less of the brominated acid. Therefore, a cartridge purification followed by reformulation could be sufficient, completely avoiding the semi-preparative HPLC purification.

The goal was to produce high specific activity. Also, instead of producing enantiomerically pure compounds, it was first sought first to obtain and evaluate the racemic mixtures and determine whether enantiomeric separations and identifications were possible. Enantiomeric separations of these small stereoisomers are notoriously difficult, and often Copper ligand exchange chiral columns is the method of choice. Nevertheless and for comparison purposes we produced here 2(L)-[¹⁸F]FP following this strategy and results are discussed further ahead.

Beyond the confirmed unstable products (Table 2), 10 compounds were synthesized and tested in vivo, comprised of 5 radiolabeled fatty acids and their corresponding ethyl esters. The data thus far demonstrates high radiolabeling yields, achieved in a 45-minute long, single pot synthesis, with a final HPLC purification. Whenever possible, hydrolyzed products were separated our using either in pure saline, or <10% ethanol/saline solutions ready for injection as HPLC mobile phases. Esters required separation in a Water/MeCN system but were easily reformulated for injection by trapping in an HLB Sep-Pak Plus (Waters, USA) and eluting the product in a <10% ethanol/saline injectable solution. All these syntheses can be easily automated in GMP synthetic units available in PET radiopharmacies for the production of [¹⁸F]FDG and other common drugs (e.g. Neptis). If needed, 3D-printed ASUs tailored to the specific production of these compounds. Our synthetic yields are on par with those obtained for the clinical production of [¹⁸F]FDG.

### Dynamic PET Imaging of FAs

Testing was initiated with the radiolabeled butyrate analogues. Because these had never been tested before, it was decided to perform dynamic PET images first to explore the biodistribution of these compounds (FIG. 11). 4-[¹⁸F]FB and 2-[¹⁸F]FB behaved very differently in vivo. The specific brain/central nervous system (CNS) uptake of 2-[¹⁸F]FB (the highest of the studied series) was intriguing from the beginning. The findings led to further exploration of static images, to avoid animal anesthesia during injection and biodistribution, either of which could hinder CNS uptake of [¹⁸F]FDG, for example. Fortunately, a stable biodistribution was observed for both compounds at around 45 minutes p.i . Blood pool activity had not completely cleared by that point, but had reduced substantially. Further, it was considered a reasonable and appropriate timepoint for these compounds and the others described for practical reasons owing to the half-life of fluorine-18. Interestingly, the brain uptake of 2-[¹⁸F]FB remained unchanged with or without isoflurane anesthesia, and with or without fasting which contrasts with using [¹⁸F]FDG.

Another interesting finding in these images was the seemingly specific gut uptake of 2-[¹⁸F]FB. Usually when compounds are eliminated via hepatobiliary excretion, the activity can be seen flowing from the gallbladder to the intestines with further downward movement from there over time. This was not the case here, and an increase in this unknown but rather specific abdominal location was noticed in time.

### Static PET Imaging:

Because of the interesting brain uptake observed during the dynamic images, and to avoid confounding artifacts, we changed our subsequent administration method from retroorbital to lateral tail-vein injection. Since our compounds are metabolic agents, we started by comparing the images of 4-[¹⁸F]FB and 2-[¹⁸F]FB with fed and fasted animals at the previously determined 45 minutes p.i. time. A slight decrease in metabolic rate was detected in fasted animals, and perhaps more importantly, a slight increase in circulatory activity with a reduction in the aforementioned specifically located gut uptake of 2-[¹⁸F]FB. Despite the small change in metabolic rate during fasting, the general biodistribution was similar to that when fed (except for the before mentioned specific gut uptake of 2-[¹⁸F]FB). Therefore, the rest of the studies were performed with fed animals to better evaluate metabolic activity of these imaging agents.

Studies performed with the corresponding ethyl esters of 4-[¹⁸F]FB and 2-[¹⁸F]FB (e-4-[¹⁸F]FB and e-2-[¹⁸F]FB) showed almost identical biodistribution, but with a slightly longer blood circulation. In fact, the increased blood half-life was a repeated trait for all studied esters and their corresponding acids, therefore delaying their final metabolic fate. We were not surprised by this behavior as similar strategies have often been pursued by the pharmaceutical industry to produce ester pro-drugs with prolonged circulation times and increased bioavailability while presenting similar biological effects to their acid counterparts. One notable example is the use of Enalapril to increase absorption and produce the Enalaprilat ACE inhibitor in vivo after hydrolysis. Similar strategies can be followed with methyl or propyl esters instead. It should be noted that myocardial uptake was not detected for any of the probes in the imaged conditions.

Interestingly, similar results to those of 2-[¹⁸F]FB were obtained with both 2-[¹⁸F]FP and [¹⁸F]FA, with decreased circulation times but maintained CNS uptake shown by 2-[¹⁸F]FB. Specific gut uptake increased as the chain length decreased, showing the highest uptake for [¹⁸F]FA. As with previous results, the corresponding esters 2-[¹⁸F]FP, 2-[¹⁸F]FIB, and [¹⁸F]FA showed similar biodistributions with increased circulation times. The non-specificity of 2-[¹⁸F]FIB in these wild-type immunocompetent animals, with a long circulation time but without defluorination could result in high lesion to background ratio and useful clinical diagnosis images. This finding could be especially useful to identify less aggressive cancers such as those of the prostate, many of which cannot be imaged with [¹⁸F]FDG as they are less anaerobic than many cancers. The finding could have additionally interesting use for brain lesions.

2-[¹⁸F]FP showed specific uptake in brain and the central nervous system, and only minor defluorination was observed. Although we have not performed a metabolite analysis, the compound seems to be stable in vivo in the studied period. 2-[¹⁸F]FP resembles lactate, a key substrate in human metabolism and especially important in both brain and cancer metabolism. However, the only metabolic fate available to lactate in vivo is its widespread conversion to pyruvate. 2-[¹⁸F]FP, on the other hand, cannot undergo the pyruvate transformation and should therefore accumulate in the target tissue (Scheme 2). This trapping phenomenon and the lack of metabolic transformation is desired in imaging agents and represents, as discussed earlier, the working principle of [¹⁸F]FDG, which traps in glucose avid tissues.

Scheme 2. In vivo metabolism. A: lactate metabolism, and B: the impossibility of 2-[¹⁸F]FP to be metabolized.

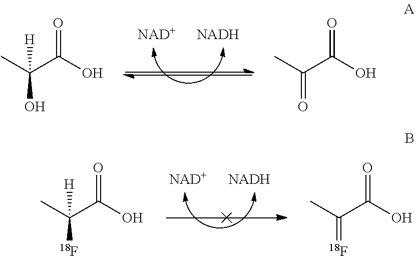

The unique characteristics of 2-[¹⁸F]FP open many possibilities to study cancers and brain/CNS function and metabolism. As with 2-[¹⁸F]FB the enantiomers have not been separated for either production or quality control. However, parametric production of 2-(L)-[¹⁸F]FP was possible since the ethyl(2R)-2-{[(4-methylphenyl) sulfonyl]oxy}propanoate is commercially available. This compound's ability to resemble (L)-lactate has been contested in vitro, with confounding in vivo results. For comparison we co-injected 2-[¹⁸F]FP separately with 0.5 and 5 mg/kg (L) and (D) sodium lactate (an increase of 5 and 50% respectively in homeostatic lactate circulation), and 5 mg/Kg of AZD-3965, an monocarboxylate transporter (MCT) 1 and 2 inhibitor (Kᵢ(MCT1)=3 nM, Kᵢ(MCT2)=20 nM) in the same animal model. AZD-3965 is advertised as an specific MCT1 inhibitor but the reality is that such a marginal difference in inhibition with MCT2, although easy to implement in vitro, is almost impossible to implement in vivo to selectively inhibit MCT1 and not MCT2.

In future experiments we will aim to titrate the correct co-administration of AZD-3965 to inhibit MCT1 receptors (gut uptake, cancer model) without inhibiting MCT2 receptors (brain), if at all possible (FIG. 2).

As it can be appreciated from FIG. 2, there was a slight increase in average brain uptake for both the fasting conditions and co-injection with 5 mg/kg of (L)-lactate, although differences are not significant. In contrast with targeted probes, metabolic probes can be found in great concentrations in homeostatic conditions (1 mM for (L)-lactate, 60) and co-injections can sometimes drive up metabolic activity. Abdominal uptake remained stable and not affected by the different imaging conditions. Once again, no defluorination was observed for 2-[¹⁸F]FP in any of the performed images, and although a metabolite analysis was not performed, these preliminary experiments showed high apparent probe stability in vivo. In contrast with the reported in vitro results, AZD-3965 blocked the overall biodistribution of 2-[¹⁸F]FP (which of course includes 2-(L)-[¹⁸F]FP) and produced almost complete excretion at the studied timepoint. Therefore, our results challenge in vivo the validity of Van Hée et. al.'s in vitro experiments, that 2-[¹⁸F]FP “does not behave as a lactate tracer” because “it does not accumulate in a MCT1-dependent manner”. Our observations instead show that 2-[¹⁸F]FP retains some of the characteristics of (L)Lactate, while also conserving its fatty acid nature. In context, the study of 2-[¹⁸F]FP in other cancer and metabolic models as a lactate surrogate is therefore warranted.

### Conclusions

High yield syntheses of 5 short-chain ¹⁸F-fluorinated fatty acids and their corresponding ethyl esters were performed using automated and commercially available techniques. All precursors used are low cost and commercially available. We have also prepared the stable fluorinated standards to ensure proper identification and quality control of the drug products.

Most of the products (n=10) were stable in saline injectable solution for over 4 hours. These stable radiolabeled molecules were tested in vivo, and results showed marked distribution differences between isomers, as well as different fatty acid chain length. Biodistribution experiments provided quantitative confirmation of the imaging results. Different imaging conditions were tested to identify in vivo behavior of several compounds. Special attention was placed on 2-[¹⁸F]FB and 2-[¹⁸F]FP, which showed marked brain uptake in contrast to their branched congener 2-[¹⁸F]FIB. If this brain uptake is a characteristic of 2-position fluoro-substituted unbranched fatty acids, it will need to be tested with longer, medium size FAs in future work. The high in vivo stability and lactate mimic of 2-[¹⁸F]FP is interesting because such a probe could provide a means to evaluate lactate metabolism in a wide variety of highly prevalent diseases, including well differentiated cancers.

### Example 2: Saturated ¹⁸F Labeled Medium Fatty Acids for PET Imaging

### Materials and Methods

### Precursors and Standards:

All 2- and n-brominated esters (radiolabeling precursors) were purchased from commercial vendors. Standards and radiolabeled compounds were synthesized using the same procedures, namely via nucleophilic substitution of cold [19F]KF spiked with 740-1850 MBq (20-50 mCi) of [¹⁸F]KF and subsequent acid hydrolysis.

### Radiolabeling Strategy:

Radiolabeling was performed by nucleophilic substitution of bromine containing ethyl esters. Briefly, [¹⁸F]F⁻ in [¹⁸O]H₂O water was received from the cyclotron (NCM USA, Bronx, NY), to which 1 ml of an 80% MeCN:H₂O solution (v:v) containing 10 mg of Kryptofix (K₂₂₂) and 5 mg of K₂CO₃ were added. The mixture was heated at 95 degrees Celsius under a light N₂ stream and house vacuum with a cold trap while shaking. When dried, two subsequent additions of 1 ml of extra-dry AcroSeal® (Acros Organics, USA) were made with an insulin syringe (BD Micro-Fine™, USA) under the same N₂ flow, vacuum and continuous heating. Once dried, the N₂ and vacuum needles were withdrawn from the silicon septum on the sealed Reacti-Vial (Pierce, USA). To this vial, 0.6 ml of a solution containing 10 mg/ml of the desired brominated precursor in dry MeCN were added and the reaction was held for 10 minutes (Scheme 3). In the case of 2-[¹⁸F]FPalmitate, (FPalm) only the hydrolized bromo-substituted acid was commercially available, so we proceeded to alkylate the acid with iodoethane. For production of 16-[¹⁸F]FPalm a commercially available methyl 16-bromopalmitate precursor was used. When preparing the ethyl esters (C5 to C8), this solution was injected into a Semi-Prep HPLC system with a PRP-1 (10×250 mm) polymeric reverse phase column (Varian-Agilent, USA) with a Water/MeCN binary system bearing 0.05% TFA. The products were collected and reformulated by trapping in a C-18 Plus Sep-Pak (Waters, USA), and eluting with a combination of EtOH and saline to form a 9% EtOH, 91% saline injectable solution. To prepare the acids, hydrolysis was performed by adding 0.6 ml of 6M HCl and heating at 95° C. for 10 minutes (Scheme 3). The hydrolyzed solutions containing FAs were purified and reformulated as explained before (using an HLB Plus cartridge instead of a C-18 Plus for 2-[¹⁸F]FV). Quality control was performed in an analytical HPLC with a reverse phase C-18 column (4.6×50 mm) and a H₂O/MeCN (0.05% TFA) 10-minute gradient at 2 ml/min flowrate. The 0.22 μm filters used were assumed integral and not bubble point tested. Endotoxins were tested by LAL clothing with a limit of 0.05 EU/ml.

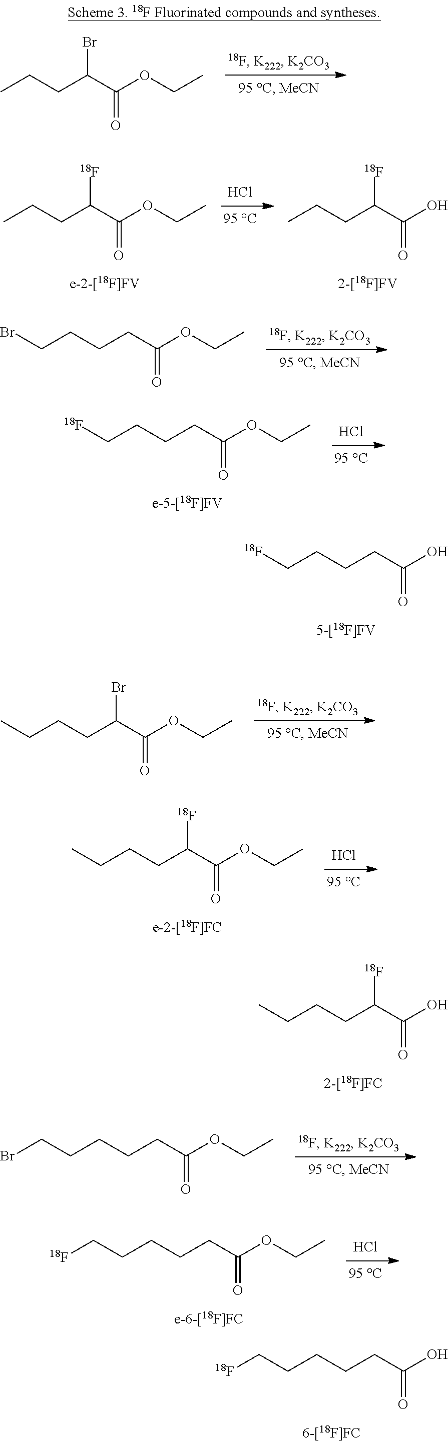

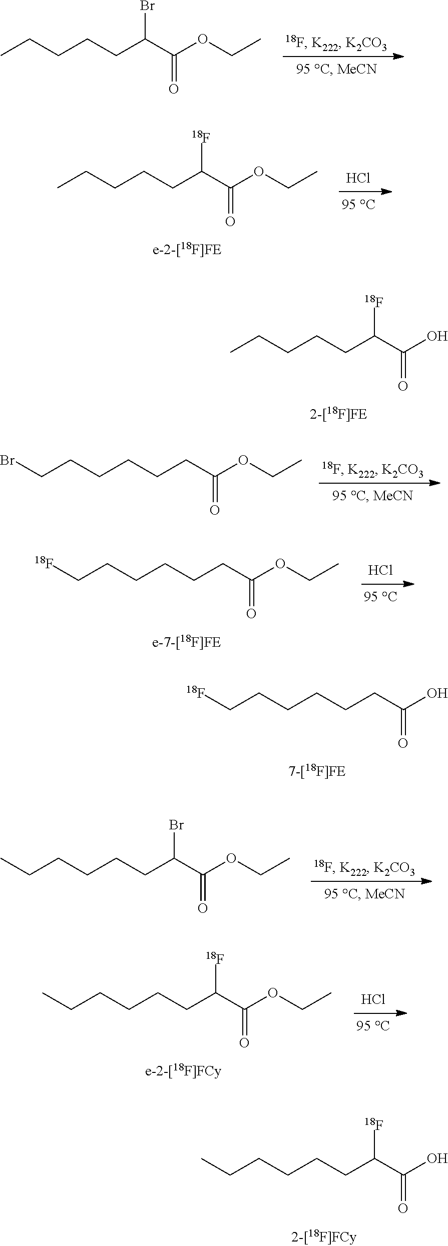

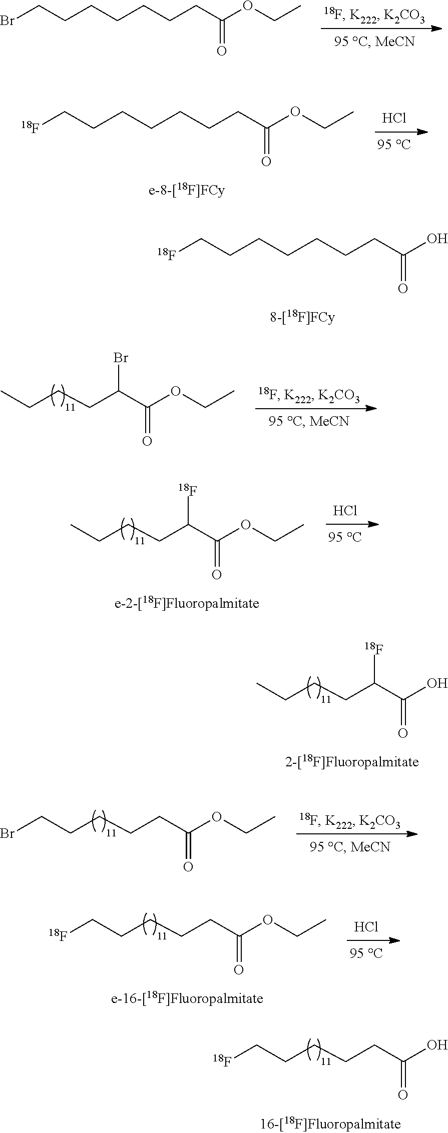

### Animal Experiments:

All animal experiments were approved by the Albert Einstein College of Medicine IACUC. Wild type immunocompetent animals were used for imaging. Feeding and water conditions were maintained throughout the study. Animals were never fasted.

### PET Imaging:

The agent was administered to awake animals via tail-vein injection (9.25-18.5 MBq, or 250-500 μCi, n=2) and placed back into their cages. At 40 minutes post injection (henceforth p.i.), animals were place in an acrylic enclosure under 4% isoflurane anesthesia in O₂ for initial knockdown, then reduced to 2.5%. Once asleep, animals were placed in the imaging holder and a 5-minute CT followed by a 15-minute static PET were performed (Approx. 45-60 min p.i.). As before, animals were allowed to recover from the anesthesia before returning to the holding facility for radiotracer decay. Images were analyzed by drawing regions of interest (ROIs) and quantifying the amount of tracer present as percent of injected activity per cubic centimeter (% IA/cc). Blood, urinary bladder and gut activity were determined by computer drawing of 2 mm diameter ROI spheres in the left cardiac ventricle, the urinary bladder and the areas of specific gut uptake, respectively. Brain, liver and kidney ROIs were drawn CT-aided and the radioactive contents were later quantified. Results are present in both μPET/CT imaging, and graphed as % IA/cc vs. compound per organ. Data from a previous publication was included for comparison.

### Results

All syntheses produced a 72.8±10.0% decay corrected yield (n=23 total, n=1-2 per compound) in a 45 minute non-optimized, one-pot synthetic scheme. Detailed data for each synthesis is presented in Table 3. In general, around 5-10% yield was lost during hydrolysis with an average 70.9±11.9% and 80.8±4.7% RCY for acids and esters respectively. All products were successfully identified with their stable analogues by HPLC UV co-registration at 220 nm and/or NMR data. All 2-position substituted compounds are racemic mixtures; separation and characterization of individual enantiomers was not attempted. A compendium of the synthetic results is detailed in Table 3.

TABLE 3
Synthetic results.

| | | | | |
|---|---|---|---|---|
| 2-[¹⁸F]FV | 2-Fluorovalerate | 72.8 ± 5.4 | 99.9 ± 0.1 | 3 |
| e-2-[¹⁸F]FV | Ethyl 2- | 83.3 | 99.7 | 1 |
| | Fluorovalerate | | | |
| 5-[¹⁸F]FV | 5-Fluorovalerate | 69.5 ± 10.2 | 99.7 ± 0.2 | 2 |
| e-5-[¹⁸F]FV | Ethyl 5- | 85.8 | 99.0 | 1 |
| | Fluorovalerate | | | |
| 2-[¹⁸F]FC | 2-Fluorocaproate | 69.1 ± 8.1 | 100.0 ± 0.0 | 4 |
| e-2-[¹⁸F]FC | Ethyl 2- | 76.4 | 99.9 | 1 |
| | Fluorocaproate | | | |
| 6-[¹⁸F]FC | 6-Fluorocaproate | 68.9 ± 1.3 | 99.9 ± 0.1 | 2 |
| e-6-[¹⁸F]FC | Ethyl 6- | 86.0 | 99.9 | 1 |
| | Fluorocaproate | | | |
| 2-[¹⁸F]FE | 2-Fluoroenanthate | 68.9 ± 9.8 | 99.9 ± 0.1 | 3 |
| e-2-[¹⁸F]FE | Ethyl 2- | 75.7 | 98.1 | 1 |
| | Fluoroenanthate | | | |
| 7-[¹⁸F]FE | 7-Fluoroenanthate | 70.5 ± 8.1 | 99.9 ± 0.2 | 2 |
| e-7-[¹⁸F]FE | Ethyl 7- | 84.9 | 99.7 | 1 |
| | Fluoroenanthate | | | |
| 2-[¹⁸F]FCy | 2-Fluorocaprylate | 69.7 ± 7.7 | 99.9 ± 0.2 | 3 |
| e-2-[¹⁸F]FCy | Ethyl 2- | 75.5 | 98.1 | 1 |
| | Fluorocaprylate | | | |
| 8-[¹⁸F]FCy | 8-Fluorocaprylate | 65.4 ± 1.2 | 100.0 ± 0.0 | 2 |
| e-8-[¹⁸F]FCy | Ethyl 8- | 78.4 | 100.0 | 1 |
| | Fluorocaprylate | | | |
| 16-[¹⁸F]FPalm | 16-Fluoropalmitate | 54.9 ± 4.0 | 99.9 ± 0.2 | 3 |
| 2-[¹⁸F]FPalm | 2-Fluoropalmitate | 41.3 ± 16.3 | 99.4 ± 0.2 | 3 |
| TOTALS | | 72.6 ± 9.6 | 99.6 ± 0.6 | 35 |

DCRCY: Decay Corrected radiochemical yield; and
RCP: Radiochemical Purity

All purified products (n=18) had an average radiochemical purity of 99.6±0.6 (Table 3), a chemical purity greater than 95% (by HPLC UV at 220 nm) and a molar activity greater than 5 Ci/μmol. Compound purity remained unchanged while kept in the injectable solution at room temperature for over 4 hours after the end of synthesis for all isolated products. The sterile, pyrogen free and isotonic products collected from the Saline/EtOH HPLC system or reformulation were filtered through a 0.2 μm membrane before injection into the animals.

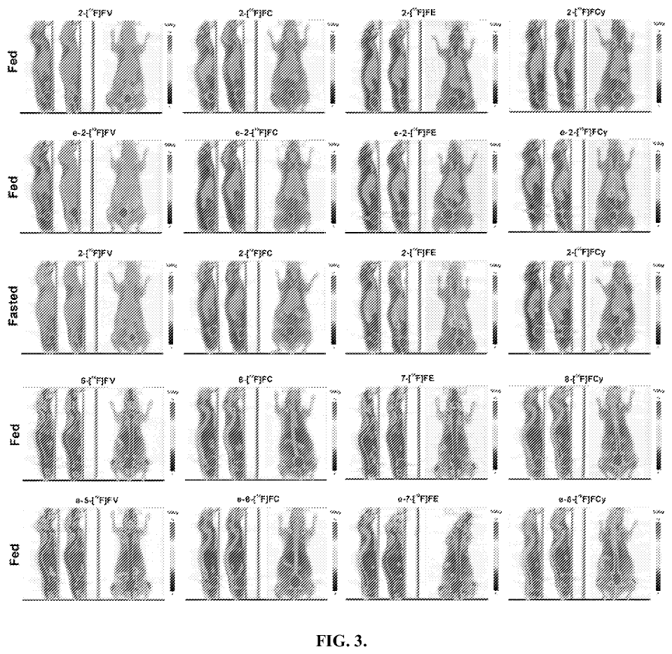

Static imaging at 45-minute pi. with fed animals, showed marked metabolic differences between conformational isomers with variable defluorination rates (FIG. 3). No difference was observed between esters and their corresponding acids, evidencing fast in vivo hydrolysis. The difference in metabolic rates between n- and 2-substituted compound is substantial but its significance was not tested. A higher metabolic rate was consistently found for n-substituted odd chain FAs (FV and FE) when compared to those of even chain (FC and FCy), but differences are barely observable for the 2-substituted compounds. Almost no defluorination was observed for 2-[¹⁸F]FC and 2-[¹⁸F]FCy, while a small bone uptake can be noticed for both 2-[¹⁸F]FV and 2-[¹⁸F]FE at the studied timepoints (FIG. 3). A slightly increased retention can be observed for fasted animals when compared to their fed scans but the overall distribution remained unchanged.

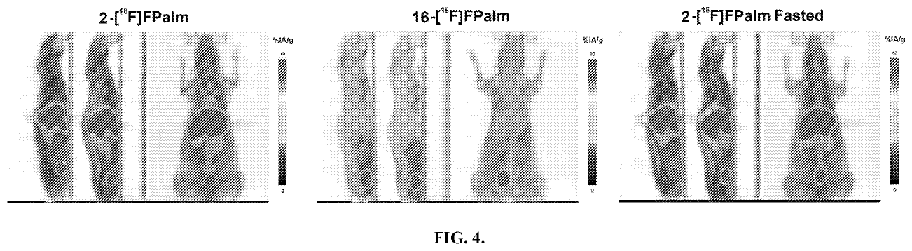

Similar scans were obtained for both 16- and 2-[¹⁸F]FPalm and it can be seen in FIG. 4. In contrast to MCFAs, radiolabeled palmitate showed marked liver and brown fat uptake in fed animals, hallmark of energy storage. The brown fat uptake was stopped when fasted, changing evidencing a metabolic change from energy storage to energy consumption. Defluorination was faster for the 16-substituted compound, although much slower in contrast to its MCFA n-substituted congeners. In contrast, a slightly higher defluorination rate can be observed for 2-[¹⁸F]FPalm when compared to its MCFA congeners. When imaging with 16-[¹⁸F]FPalm a bright spot was found in one the animal's liver, but later autopsy did not revealed any interesting finding.

Slow renal excretion was observed for all compounds. High brain uptake, also shown by [¹⁸F]FA, 2-[¹⁸F]FP and 2-[¹⁸F]FB can be observed for 2-[¹⁸F]FV and 2-[¹⁸F]FC, but it is significantly reduced for 2-[¹⁸F]FE, 2-[¹⁸F]FCy and 2-[¹⁸F]FPalm (FIGS. 3, 4 and 5). a specific abdominal uptake, evident in 2-[¹⁸F]FV and its smaller congeners is also not found for 2-[¹⁸F]FC, 2-[¹⁸F]FE, 2-[¹⁸F]FCy and 2-[¹⁸F]FPalm (FIGS. 3 and 4). Liver uptake is reduced in a remarkable linear fashion with increasing chain length as heart and kidney activities are increased due to an apparent increase in albumin binding and therefore blood content. Interestingly this blood content was reduced for 2-[¹⁸F]FPalm resulting in predominant liver uptake.

### Discussion

### Radiolabeling Strategy:

Designed to enable easy automation, these FA compounds were produced in high yields. To avoid fluoride elimination, a 5/10 mg/ml K₂CO₃/K₂₂₂ system was used, which was identified as the lowest amount of base that can completely elute a fluorine loaded QMA cartridge. Nucleophilic substitution of brominated precursors provides an easy and low-cost alternative to other, more complex precursors. The 2-substituted enantiomers here produced were not characterised. However, 18 compounds were prepared and tested in vivo, comprised of 10 radiolabeled fatty acids and most of their corresponding ethyl esters. The syntheses of their corresponding methyl and propyl esters behave very similarly. The data demonstrates high radiolabeling yields in a 45-minute long, single pot synthesis, with a final HPLC purification. Although HPLC purification was used to separate the fluorinated products from their corresponding brominated analogues, future production of these FAs can be simplified by performing a cartridge purification directly since the minute amounts of brominated analogues present will neither decrease the specific activity nor produce any interference in the imaging process.

### PET Imaging:

PET imaging results deserve substantial discussion. First, all n-substituted compounds studied underwent fast degradation and defluorination in vivo when compared to all 2-substituted compounds. This difference in metabolic rate is somewhat reduced for the longest chain studied, although no difference is observed for the MCFAs. A metabolic difference can be found between odd and even n-substituted MCFAs, with the odd chain 5-[¹⁸F]FV and 7-[¹⁸F]FE undergoing a much faster metabolic rate than that of 6-[¹⁸F]FC and 8-[¹⁸F]FCy. Such differences are eliminated with α-fluorinations with near-complete β-oxidation stoppage.

The possibility of stopping β-oxidations with α-fluorinations is important. Stopping (or delaying) β-oxidation with minimal modifications of fatty acids has long been an unachieved goal. It is well documented in the literature that stopping β-oxidation often require drastic molecular changes including S for C substitutions, phenyl additions, and aliphatic chain branching. The probes presented here open new imaging possibilities, seldom achieved with other methods, and with a simple F for H exchange. These probes could be applied to cardiac, cancer, metabolism, and inflammation imaging applications, among many others. However, these compounds constitute an important, practical alternative to carbon-11 labeled fatty acids, as these undergo β-oxidations as part of the central carbon metabolism rapidly producing ¹¹CO₂ and other metabolic substrates.

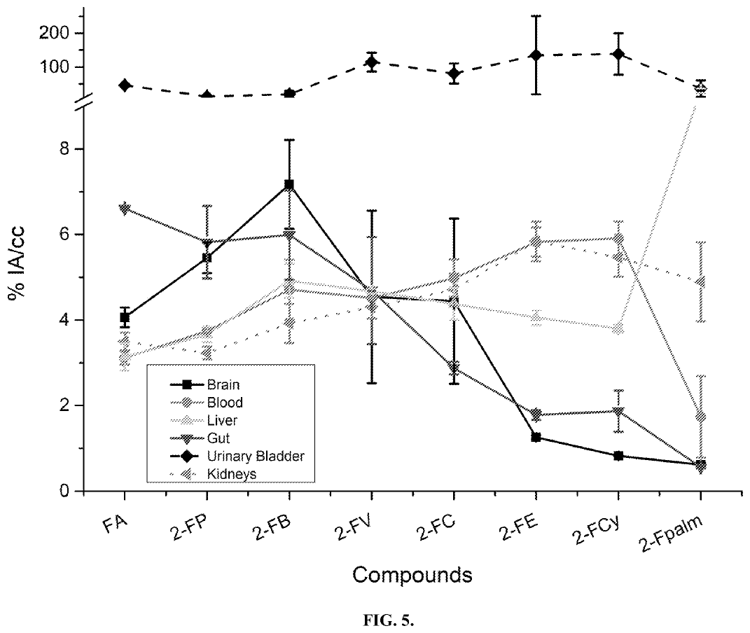

Another interesting finding is related to brain metabolism and its selectivity. All saturated α-fluorinated SCFAs tested in our laboratory had a significant brain uptake, higher than all other observed specific uptake and peaking for 2-[¹⁸F]Fluorobutyrate (FIG. 5). Interestingly all brain uptake is stopped with 2-[¹⁸F]FE and beyond, as 2-[¹⁸F]FC is the longest substrate absorbed by the brain.

Albumin binding increased in an almost linear fashion from Acetate to Caprylate but was significantly reduced with palmitate. In addition, liver and brown fat uptake with palmitate was not observed with any of the MCFAs.

### Conclusions

High yield syntheses of 8 medium chain and 2 long chain n and α-[¹⁸F]fluorinated fatty acids and their corresponding ethyl esters were performed using commercially available precursors. All products (n=10) were stable in saline injectable solution for over 4 hours. Testing in vivo showed biodistribution differences between conformational isomers and fatty acid chain length. Metabolic differences were also found between odd and even chain FAs. The data show that β-oxidations can be stopped (or significantly delayed) with a simple α-fluorination regardless of the chain length, which opens new possibilities for imaging in various medical applications. It was also found that brain uptake is dependent on chain length and stops abruptly with 2-[¹⁸F]FE, being 2-[¹⁸F]FC the longest substrate absorbed.

### Example 3. 18F-Labeled Fatty Acids Vs [¹⁸F]FDG in a Preclinical Ovarian Cancer Model

Imaging was performed with well differentiated ovarian adenocarcinoma model (SKOV3) with two α-fluorinated fatty acids and compared to the commercially available [¹⁸F]FDG.

### Materials and Methods

### Radiolabeling Strategy:

All precursors were purchased from commercial vendors and are readily available. All radiosyntheses, standards, purifications, and quality control procedures were performed as described in Example 1.

### Animal Experiments:

All animal experiments were approved by the Albert Einstein College of Medicine IACUC. Nude mice were xenografted with 10M SKOV3 cells in Matrigel in the right upper shoulder. Tumors were allowed to grow for 3 weeks until a palpable mass was observed (100-400 mm³). This animal model was used, both for imaging and biodistribution experiments.

### μPET/CT Imaging:

To anesthetized animals (3% isoflurane in 97% O₂), [¹⁸F]-labeled compounds were injected in the retroorbital cavity as a high specific activity saline solution (<10% EtOH, 9.25-18.5 MBq, or 250-500 μCi, n=2) and immediately placed in a Siemens Inveon μPET/CT camera (Siemens, USA) for dynamic imaging. After the initial 60 minutes animals were placed back in their cages and allowed to recover from the anesthesia. Subsequent static 15 minute μPET/CT imaging was performed at 2 and 4 hours post administration (p.i.). At the end of the study, animals were allowed to recover from anesthesia and were carefully observed before moving them back to the holding facility for decay. Dynamic data was reconstructed as twelve 5-minute frames (5-65 minutes p.i.). Regions of interest (ROIs) were drawn aided by CT for brain, kidneys, liver and tumor. Blood and urine contents were obtained by drawing 2 mm diameter spheres in left ventricle and the urinary bladder respectively. Muscle and bone contents were determined by drawing 2 mm diameter spheres in the thigh and knee joint, respectively. ROIs were quantified and a time activity curve expressed as percent injected activity per cubic centimeter (% IA/cc) vs. time was obtained.

### Organ Biodistribution Studies:

To better quantify organ contents, selected radiopharmaceuticals were studied in an organ biodistribution. For these studies the activity was administered vial tailvein injection in awake animals (1.5 MBq, 40 μCi, n=3-4) and placed back in their respective cages. At 55 minutes p.i., animals were placed into an acrylic enclosure and anesthetized with 4% isoflurane in O₂ for 5 minutes. At 60 minutes p.i. animals were euthanized by neck dislocation under isoflurane anesthesia. Organs including blood, heart, lungs, liver, intestines (with contents), stomach, spleen, pancreas, kidneys, bone, muscle, brain and tumors were collected, weighed and counted in a Packard automated well counter (Perkin Elmer, USA). Results were expressed as percent of injected activity per gram of tissue (% IA/g).

### Results

### Radiolabeling

All compounds were produced as reported in a radiochemical yield ranging from 65-85% and a radiochemical purity >98%, a chemical purity greater than 95%, and a molar activity greater than 5 Ci/μmol. As reported, compound purity remained unchanged while kept in the injectable solution at room temperature for over 4 hours after the end of synthesis for all isolated products. Sterile, pyrogen free and isotonic products (in saline solution with less than 10% EtOH) were injected into the animals.

### μPET/CT Imaging

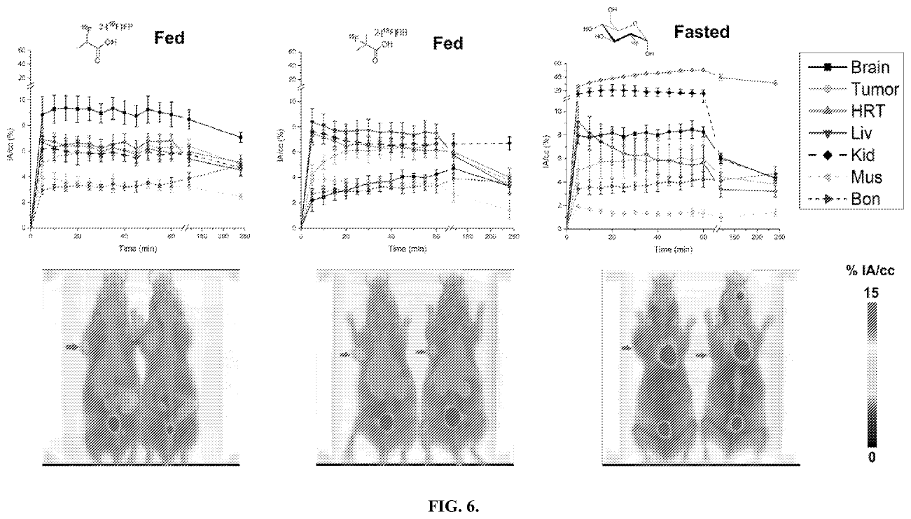

Results for the μPET/CT imaging can be found in FIG. 6. Each of 2-[¹⁸F]FP, 2-[¹⁸F]FIB, and [¹⁸F]FDG was administered to the mouse and imaging was performed on regions of interest (ROIs), for example, the brain, kidneys, liver and tumor.

### Organ Biodistribution Studies:

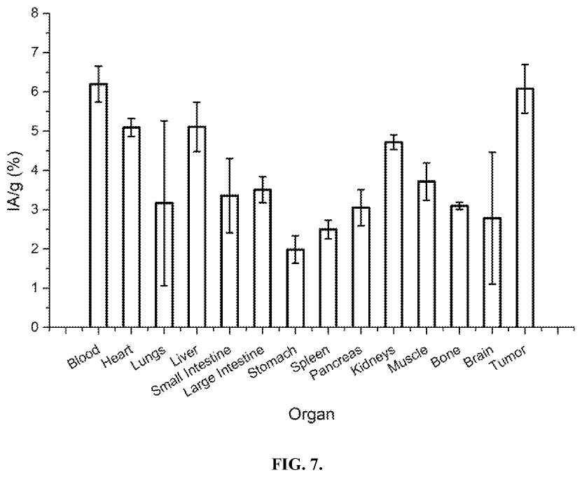

Results for the organ biodistribution studies of 2-[¹⁸F]FIB can be found in FIG. 7.

### Other Notable Biodistribution Notes:

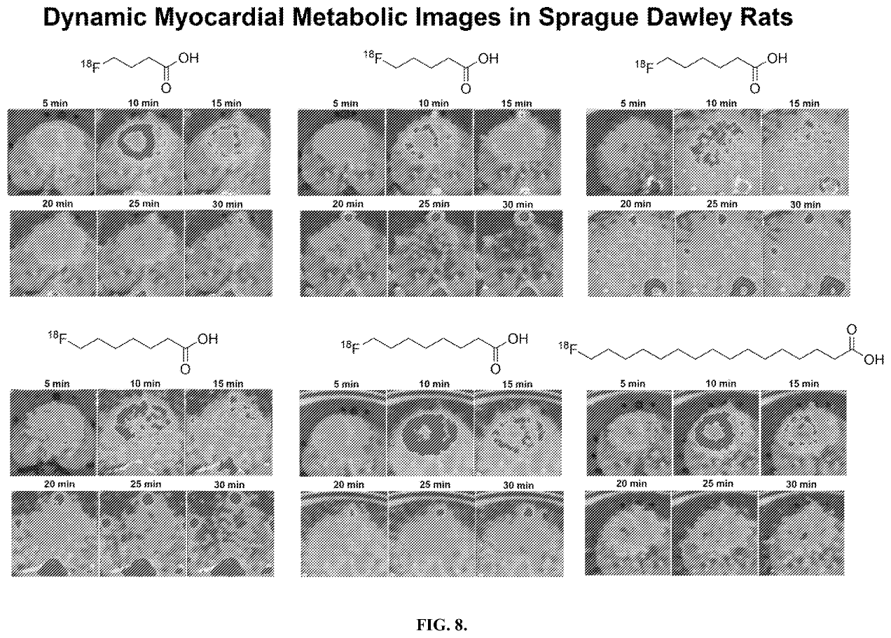

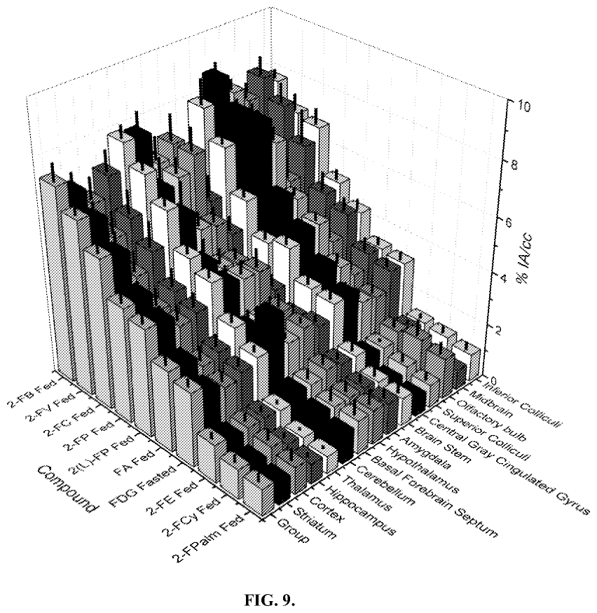

Cardiac studies are possible with n-[¹⁸F]FAs. These probes are metabolized in the myocardium and therefore present a strong uptake in normally fed animals, shown in FIG. 8. Similarly, 2-[¹⁸F]FAs' brain metabolism is of interest and shown in FIG. 9. Their uptake is higher in fed animals than that of [¹⁸F]FDG in fasted animals in most regions of the brain.

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A compound which is a fatty acid, or a salt thereof, or a fatty acid ester comprising a fluorine-18, provided the compound is not [18F]-fluoroacetate or 2-[18F]fluoropropionate, or a salt thereof.

2. The compound of claim 1, wherein the fatty acid or salt thereof or fatty acid ester is a saturated fatty acid or fatty acid ester substituted with a fluorine-18.

3. The compound of claim 1 or 2, wherein the fatty acid or salt thereof or fatty acid ester is a branched fatty acid or salt thereof or fatty acid ester substituted with a fluorine-18.

4. The compound of any one of claims 1-3, wherein the fatty acid or salt thereof or fatty acid ester is a short-chain fatty acid or salt thereof or fatty acid ester.

5. The compound of any one of claims 1-3, wherein the fatty acid or salt thereof or fatty acid ester is a medium-chain fatty acid or salt thereof or fatty acid ester.

6. The compound of any one of claims 1-3, wherein the fatty acid or salt thereof or fatty acid ester is a long-chain fatty acid or salt thereof or fatty acid ester.

7. The compound of any one of claims 1-3, wherein the fatty acid or salt thereof or fatty acid ester is a very long-chain fatty acid or salt thereof or fatty acid ester.

8. The compound of any one of claims 1-7, wherein the carbon alpha to the carbonyl is substituted with a fluorine 18.

9. The compound of any one of claims 1-7, wherein the terminal carbon is substituted with a fluorine 18.

10. The compound of any one of claims 1-9 having the structure of Formula I: 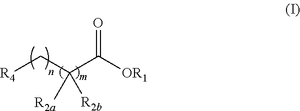
wherein
n is 0 to 15;
m is 0 or 1;
R₁ is H or alkyl;
R₂ₐ is H, alkyl, or ¹⁸F;
R_{2b} is H or alkyl;
R₄ is H or C(R₄ₐ)(R_{4b})(R_{4c}); and
R₄ₐ, R_{4b}, and R_{4c} are each independently H, alkyl, or ¹⁸F,
provided that one and only one of R₂ₐ, R₄ₐ, R_{4b}, and R_{4c} is a ¹⁸F,
or a salt thereof.

11. The compound of claim 10, wherein
R₁ is H or (C₁-C₆)alkyl;
R₂ₐ is H, (C₁-C₆)alkyl, or ¹⁸F;
R_{2b} is H or (C₁-C₆)alkyl;
R₄ is H or C(R₄ₐ)(R_{4b})(R_{4c}); and
R₄ₐ, R_{4b}, and R_{4c} are each independently H, (C₁-C₆) alkyl, or ¹⁸F; or salt thereof

12. The compound of claim 10 or 11, wherein
R₁ is H, CH₃, or CH₂CH₃;
R₂ₐ is H, CH₃, or ¹⁸F;
R_{2b} is H or CH₃;
R₄ is H or C(R₄ₐ)(R_{4b})(R_{4c}); and
R₄ₐ, R_{4b}, and R_{4c} are each independently H, CH₃, or ¹⁸F; or salt thereof

13. The compound of any one of claims 10-12 having the structure: 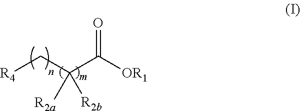
or salt thereof.

14. The compound of claim 13 or salt thereof, wherein n is 0 to 6.

15. The compound of claim 13 or 14 or salt thereof, wherein R₁ is H or CH₂CH₃; and R₄ is CH₃.

16. The compound of any one of claims 13-15 or salt thereof, wherein R₂ₐ is ¹⁸F.

17. The compound of any one of claims 13-16 having the structure: 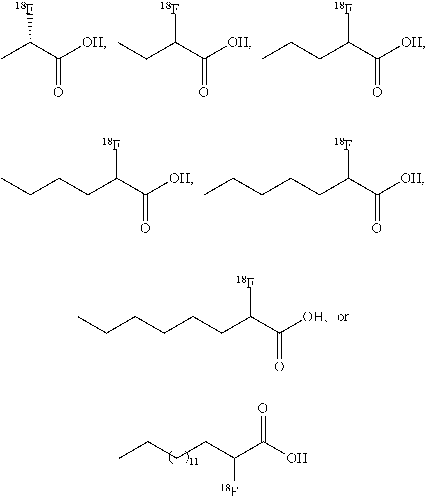
or salt thereof.

18. The compound of any one of claims 13-16 having the structure:

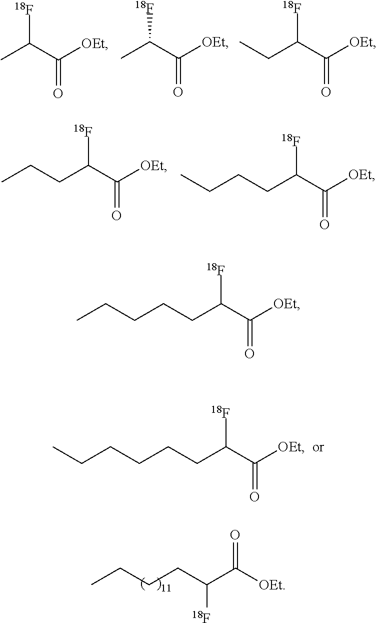

19. The compound of any one of claims 13-16 or salt thereof, wherein R₂ₐ is CH₃.

20. The compound of any one of claim 13-16 or 19 having the structure: 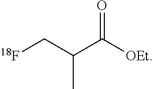
or salt thereof.

21. The compound of any one of claim 13-16 or 19 having the structure:

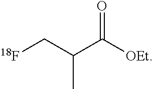

22. The compound of any one of claims 10-12 having the structure: 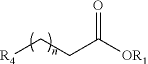
or salt thereof.

23. The compound of claim 22 or salt thereof, wherein n is 0 to 13.

24. The compound of claim 22 or 23 or salt thereof, wherein R₁ is H or CH₂CH₃.

25. The compound of any one of claims 22-24 or salt thereof, wherein R₄ is C(R₄ₐ)(R_{4b})(R_{4c});
R₄ₐ and R_{4b} are each H or CH₃; and R_{4c} is ¹⁸F.

26. The compound of any one of claims 22-25 having the structure: 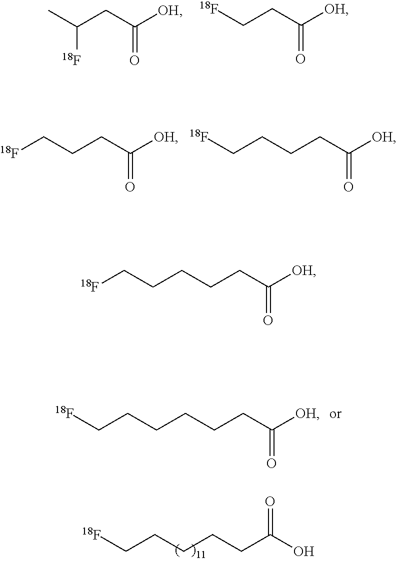
or salt thereof.

27. The compound of any one of claims 22-25 having the structure:

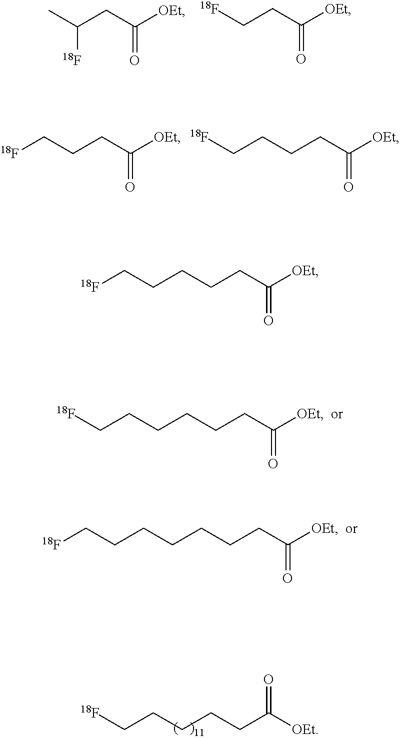

28. The compound of any one of claims 10-12 having the structure: 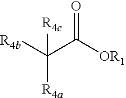
or salt thereof.

29. The compound of claim 28 or salt thereof, wherein R₁ is H or CH₂CH₃.

30. The compound of claim 28 or 29 or salt thereof, wherein R₄ₐ, R_{4b}, and R_{4c} are each independently H, CH₃, or ¹⁸F;

31. The compound of any one of claims 28-30 having the structure: 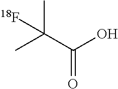
or salt thereof.

32. The compound of any one of claims 28-30 having the structure:

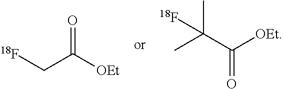

33. A composition comprising a compound or salt thereof of any one of claims 1-32; and a pharmaceutically acceptable carrier.

34. A method of imaging target cells in a subject comprising:
(i) introducing into the subject an effective amount of a compound or salt thereof of any one of claims 1-32;
(ii) detecting in the subject the location of the compound in the subject; and
(iii) obtaining one or more images of the target cells in the subject based on the location of the compound in the subject.

35. The method of claim 34, wherein the ¹⁸F in the compound is detected by positron emission tomography (PET).

36. The method of claim 34 or 35, wherein the target cell is a cancer cell, a cardiac muscle cell, a nerve cell, or a glial cell.

37. The method of any one of claims 34-36, further comprising (iv) determining whether the subject is afflicted with a cardiovascular disease, a neurodegenerative disease, or a cancer based on an analysis of the one or more images of the target cells.

38. A method of imaging cellular uptake of a fatty acid in a target cell of a subject comprising:
(i) introducing into the subject an effective amount of a compound or salt thereof of any one of claims 1-32,
(ii) performing one or more positron emission tomography (PET) scans of the subject to detect the compound in the target cell; and
(iii) obtaining one or more images of the target cell based on the location of the compound in the subject.

39. A method of imaging β-oxidation of a fatty acid in a target cell of a subject comprising:
(i) introducing into the subject an effective amount of a compound or salt thereof of any one of claims 1-32;
(ii) performing one or more positron emission tomography (PET) scans of the subject to detect the compound in the target cell; and
(iii) obtaining one or more images of the target cell based on the location of the compound in the subject.

40. The method of claim 38 or 39, wherein the ¹⁸F in the compound is detected by the positron emission tomography (PET).

41. The method of any one of claims 38-40, wherein the target cell is a cancer cell, a cardiac muscle cells, a nerve cell, or a glial cell.

42. The method of any one of claims 38-41, wherein the one or more positron emission tomography (PET) scans of the subject are performed on a tissue of interest in the subject.

43. The method claim 42, wherein the tissue of interest is cardiac tissue.

44. The method claim 43, wherein the tissue of interest is afflicted with a cardiovascular disease.

45. The method claim 42, wherein the tissue of interest is brain tissue.

46. The method claim 45, wherein the tissue of interest is afflicted with a neurodegenerative disease.

47. The method claim 42, wherein the tissue of interest is brain, breast, lung, kidney, gastrointestinal, ovary, pancreatic, prostate, or spinal cord tissue.

48. The method claim 47, wherein the tissue of interest is afflicted with a cancer.

49. The method any one of claims 42, 43, 45, and 47, wherein the tissue of interest is healthy tissue.
